SEQUENCE LISTING (1 sequence)

SEQ ID NO: 1 - DNA, 5958 bp, Mus musculus
agcgcccaatacgcaaaccgcctctccccgcgcgttggccgattcattaatgcaactatggccatttaatgtaaatacttaagaaaaaaaaccaaattaattttgatacatgctgcatgtgaaagacccccgctgacgggtagtcaatcactcagaggagaccctcccaaggaacagcgagaccacaagtcggatgcaactgcaagagggtttattggatacacgggtacccgggcgactcagtcaatcggaggactggcgcgccgagtgaggggttgtgggctcttttattgagctcggggagcagaagcgcgcgaacagaagcgagaagcgaactgattggttagttcaaataaggcacagggtcatttcaggtccttggggcaccctggaaacatctgatggttctctagaaactgctgagggctggaccgcatctggggaccatctgttcttggccctgagccggggcaggaactgcttaccacagatatcctgtttggcccatattcagctgttccatctgttcttggccctgagccggggcaggaactgcttaccacagatatcctgtttggcccatattcagctgttccatctgttcctgaccttgatctgaacttctctattctcagttatgtatttttccatgccttgcaaaatggcgttacttaagctagcttgccaaacctacaggtggggtctttcattcccccctttttctggagactaaataaaatcttttattttatcgtcgaccactgtgctggcggccgctcgagtttaaatagtacataatggatttccttacgcgaaatacgggcagacatggcctgcccggttattattatttttgacaccagaccaactggtaatggtagcgaccggcgctcagctggaatatcaccactttgtacaagaaagctgggttgatcaacagcatcactgagcttctttccccatcccaccccccacccctgttgtgcttttaatccctcctcagtaaaagaatttaaccccaaagctccaggttctcttgtctacctcccttgccttggctcacagcatccccagggagggctggtgcctcagtttgaatgcatgggagagcccagagcagtgacgggaacagagggaaaggcctcgccctcaggaaaagggactgagtagagtgtggtgaagtgggggcttccatagcctggggtgccaaagtggggacctgggggcagaggaaaggatacagccccccctgggaaaggtgtccctgtagcctcatactcttctcgttgggaatactcaatacttgatcttttgccctctggcgccggttacagaaccatactcgaaccacatccttctctagcccaagctgattggcgatgtgagtgatctgctgtagggagggcttcgggcacttcagaaacatggtctccagactccacctcacacggttctcaatgctagttcgctttctcttccgggcctgcaccagggtctccgatttgcatatctcctgaaggttctcattgttgtcggcttcctccacccacttctccagcaggggccgcagcttacacatgttcttaaggctgagctgcaaggcctcgaagcgacagatggtggtctggctgaacacctttccaaagagaacgcccagggtgagccccacgtcggcctgggtgtaccccaaggtgatcctcttctgcttcagcagcttggcaaactgttctagctccttctgcagggctttcatgtcctgggactcctcgggagttggttccaccttctccaacttcacggcattggggcggtcggcacagggctcagaggaggttccctctgagttgctttccactcgtgctcctgcctggccctcaggctgcaaagtctccacgccaacttgggggactaggcccagtccaacctgaggtccacagtatgccatccctccgcagaactcgtatgcgggcggacatggggagatccccaatacctctgagcctggtccgattccaggcccacctggaggcccttggaagcttagccaggttcgagaatccacccagcccggctccagccctgctgacccatcacccccacctggtgggggtgagaaggcgaagtctgaagccaggtgtccagccatggggaaggtggagcctgcttttttgtacaaacttgtgatttcccgtaccaccacactgggatccttaattaactagctagatccggcagtctagaggatggtccacccccggggtcggcagccttcacgtgggcggcgtgtatccaagctgcgatgccgtctactttgagggcggtgggggtggtcagcaggactgtgtaaggtcctttccagcgaggttctaggttcttagtctggtgtcggcggacccacactgtgtcgccgactcggtaagggtgaggtaccaccggtcggtccagttgttcttggtaggctgccgccagaggtctccagacttcgtgctggactaagtagagagcctgtaagtgagcttggagagaggggctgttagtaactcttgtcatgtcagggtcagggaagtttacaaggggcgggggtgccccatataagatctcatatggccatatgggggcgcctagagaaggagtgagggctggataaagggaggatcgaggcggggtcgaacgaggaggttcaagggggagagacggggcggatggaggaagaggaggcggaggcttagggtgtacaaagggcttgacccagggaggggggtcaaaagccaaggcttcccaggtcacgatgtaggggacctggtctgggtgtccatgcgggccaggtgaaaagaccttgatcttaacctgggtgatgaggtctcggttaaaggtgccgtctcgcggccatccgacgttaaaggttggccattctgcagagcagaaggtaacccaacgtctcttcttgacatctaccgactggttgtgagcgatccgctcgacatctttccagtgacctaaggtcaaacttaagggagtggtaacagtctggccctaattttcagacaaatacagaaacacagtcagacagagacaacacagaacgatgctgcagcagacaagacgcgcggcgcggcttcggtcccaaaccgaaagcaaaaattcagacggaggcgggaactgttttaggttctcgtctcctaccagaaccacatatccctcctattaggggggtgcaccaaagagtccaaaacgatcgggatttttggactcaggtcgggccacaaaaacggcccccgaagtccctgggacgtctcccagggttgcggccgggtgttccgaactcgtcagttccaccacgggtccgccagatacagagctagttagctaactagtaccgacgcaggcgcataaaatcagtcatagacactagacaatcggacagacacagataagttgctggccagcttacctcccggtggtgggtcggtggtccctgggcaggggtctcccgatcccggacgagcccccaaatgaaagacccccgctgacgggtagtcaatcactcagaggagaccctcccaaggaacagcgagaccacaagtcggatgcaactgcaagagggtttattggatacacgggtacccgggcgactcagtcaatcggaggactggcgccccgagtgaggggttgtgggctcttttattgagctcggggagcagaagcgcgcgaacagaagcgagaagcgaactgattggttagttcaaataaggcacagggtcatttcaggtccttggggcaccctggaaacatctgatggttctctagaaactgctgagggctggaccgcatctggggaccatctgttcttggccctgagccggggcaggaactgcttaccacagatatcctgtttggcccatattcagctgttccatctgttcctgaccttgatctgaacttttctattctcagttatgtatttttccatgccttgcaaaatggcgttacttaagctagcttgccaaacctacaggtggggtctttcattatgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttgcggccggccgcaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

(19) United States
(12) Patent Application Publication
(10) Pub. No.: US 2018/0037871 A1
(43) Pub. Date: Feb. 8, 2018

(54) CANCER INITIATING CELL AND USE THEREOF

(71) Applicant: Thai-Yen Ling, Taipei City (TW)

(72) Inventors: Thai-Yen Ling, Taipei City (TW); Sui-Yuan Chang, Taipei City (TW); Tai-Ling Chao, Taipei City (TW); Sing-Yi Gu, Taipei City (TW); Yufeng Jane Tseng, Taipei City (TW); Ming-Tsung Hsu, Taipei City (TW)

(21) Appl. No.: 15/230,634

(22) Filed: Aug. 8, 2016

(51) Int. Cl. C12N 7/00; C07K 14/47; C12N 15/85

(52) U.S. Cl. CPC C12N 7/00; C12N 15/85; C07K 14/47; C12N 2770/32322; C12N 2710/10022

(57) ABSTRACT

The present invention relates to a cancer initiating cell comprising an isolated coxsackievirus and adenovirus receptor positive mouse pulmonary stem/progenitor cell that overexpresses Oct-4.

### CROSS-REFERENCES TO RELATED APPLICATIONS

This application contains a Sequence Listing in computer readable form. The computer readable form is incorporated herein by reference.

### FIELD OF THE INVENTION

The present invention relates to a cancer initiating cell comprising an isolated coxsackievirus and adenovirus receptor positive mouse pulmonary stem/progenitor cell that overexpresses Oct-4, and use thereof.

### BACKGROUND OF THE INVENTION

Lung cancer is a leading cause of cancer-related death worldwide, and the overall 5-year survival rate remains less than 14%. Increasing evidence suggests that cancer stem cells, also known as cancer initiating cells (CICs), play critical roles in tumor growth and resistance to conventional chemotherapies, and may be responsible for tumor metastasis and recurrence. The cancer stem cell: premises, promises and challenges.

CICs have been identified using different in vitro assays and cell biomarkers, such as side population analysis, sphere formation assay, chemoresistance, aldehyde dehydrogenase (ALDH) activity, and the cell marker CD133. However, these in vitro assays alone are not enough to demonstrate that the identified cells are in fact CICs. Therefore, certain in vivo assays, such as limiting dilution transplantation experiments in animal models, are used to verify the results of in vitro assays. Unfortunately, studies have yielded conflicting identification of CICs in different types of cancer. The discrepancies in CICs identification may be due to the fact that the studied cells derived from different cancer cell lines or well-developed tumors. The phenotypic and functional heterogeneity of clinical tumor samples may exacerbate the difficulty in identifying CICs.

Different hypotheses have been proposed to explain the formation of CICs, such as mutations in adult stem/progenitor cells or the acquisition of stem-like characteristics in differentiated cells; however, the sources of cells and processes involved in the development of CICs remains unclear. In the K-ras^{GI2D} mutation conditional mice model, the stem cells located at the bronchioalveolar duct junction were examined as potential origin for adenocarcinoma after Cre/lox mediated activation. Another study has demonstrated that Oct-4, mediated by IGF-ER signaling, can form a complex with β-catenin and Sox-2 to play a crucial role in the self-renewal and oncogenic potential of CICs in lung adenocarcinomas. Additionally, co-expressing Oct-4 and Nanog in A549 lung adenocarcinoma cell line can control epithelial-mesenchymal transdifferentiation, regulate tumor initiating ability, and promote metastasis behavior. Moreover, a high level of Oct-4 in non-small cell lung cancer patients has been correlated with metastasis and a lower survival rate. Although these studies have demonstrated that certain pluripotent genes, Oct-4, Sox-2 and Nanog, are closely associated with tumor initiating properties, the connection between aberrant pluripotent genes expression and the generation of CICs is unclear and requires further clarification.

### BRIEF DESCRIPTION OF THE DRAWINGS

The patent or application file contains at least one drawing executed in color. Copies of this patent or patent application publication with color thawing(s) will be provided by the Office upon request and payment of the necessary fee.

FIG. 1 shows CAR+/mPSCs cultivation, isolation and differentiation. (A) Immunofluorescence staining of CAR in the epithelial colonies of primary cultures. (i), The epithelial colony is denoted by the white dotted line in the phase contrast image. (ii), Immunofluorescence images show CAR expressed at cell-cell junctions of the epithelial colony. (iii), Magnified image of the boxed area in panel A-ii. (Scale bar: 100 μm) (B) The CAR-positive population of the primary culture is identified and isolated using FACS, referred to as CAR⁺/mPSCs. (C) Gene expression profiles of CAR⁺/mPSCs are analyzed using PCR and real-time PCR. Gene expression of CAR, Oct-4, Sox-2, and Nanog are evaluated. L, mouse lung tissue; CAR⁺, CAR⁺/mPSCs; ES, mouse embryonic stem cell line (E14). Data are expressed as the mean±SD. (D) CAR⁺/mPSCs differentiation. CAR⁺/mPSCs differentiate into type-I pneumocytes for 7 d after isolation. At day 1, the magnified image shows the isolated cells in the boxed area. White dashed lines indicate the phase contrast images of the differentiated cells at day 4 and 7. The expression of CAR and type-I pneumocyte markers, T1α and AQP5, are evaluated using immunofluorescence staining. CAR expression is detected at day 1 and the magnified image of the boxed area shows CAR expression at the cell-cell junctions of isolated cells. At day 4 and day 7, CAR expression is absent. T1α and AQP5 expression are detected at day 4 and day 7. (Scale bar: 100 μm)

FIG. 2 shows overexpression of Oct-4 in CAR+/mPSCs. It is a procedure to overexpress Oct-4 in CAR⁺/mPSCs. (i), Representative phase contrast image of primary culture. The magnified image shows the epithelial colony of mPSCs. (ii), mPSCs are isolated according to CAR-positive expression of primary cultures using flow cytometry and subsequent transfection with retroviral vectors encoding Oct-4 cDNA. (iii), Transfected CAR⁺/mPSCs are co-cultivated with feeder cells at day 2. Cobblestone-like colonies are observed at day 21. The magnified image shows the morphology of one colony. (iv), Isolation and expansion of individual cobblestone-like colonies at day 28. (v), Colonies are established as cell clones, comprising C1, E9, and C7 clones. Representative morphology images of the C1 clone. (Scale bar: 100 μm)

FIG. 3 shows overexpression of Oct-4 in type-I pneumocytes. In CAR⁺/mPSCs-derived type-I pneumocytes, a time course of the Oct-4 overexpression procedure is shown. (i), Representative phase contrast image of primary cultures showing epithelial colony. (ii), CAR⁺/mPSCs undergo differentiation into type-I pneumocytes for day 7. (iii), At day 8, type-I pneumocytes are transfected with retroviral vectors encoding Oct-4 cDNA. (iv), Transfected cells proliferate upon addition of a feeder cell supplement at day 10, 21, 35, and 42. (Scale bar: 100 μm)

FIG. 4 shows Oct-4 hyperexpression in CAR⁺/mPSCs^{Oct-4}^{_}^{hi}. (i), Oct-4 expression in CAR^{|}/mPSCs and CAR^{|}/mPSCs^{Oct-4 hi} C1, E9, and C7 clones are analyzed using Western blot. ES denotes mouse embryonic stem cell line (E14). (ii), Quantification of Oct-4 expression. Data are presented as the mean±SD. **P<0.01 compared with CAR^{|}/mPSCs.

FIG. 5 shows CAR expression of CAR⁺/mPSCs^{Oct-4}^{_}^{hi}. (A) Flow cytometry is used to analyze the population of CAR-positive in C1, E9, and C7 clones. (B) Immunofluorescence staining displays CAR expression in cell membrane of C1, E9, C7 clones. CAR is labeled in Alex 488 nm fluorescence. DAPI is used as the nucleus marker. The insert image shows the magnification in square. Scale bar: 100 μm.

FIG. 6 shows phenotypic alterations in CAR⁺/mPSCs^{Oct-4}^{_}^{hi}. (A) Altered cell cycle distribution. It shows a representative cell cycle of C1 clone and CAR⁺/mPSCs. (B) CAR⁺/mPSCs^{Oct-4}^{_}^{hi} C1, E9, and C7 clones exhibit proliferation capacity. It shows a proliferation curve of CAR⁺/mPSCs and CAR⁺/mPSCs^{Oct-4}^{_}^{hi} C1, E9, and C7 clones. (C) Telomerase activity in CAR⁺/mPSCs and CAR⁺/mPSCs^{Oct-4}^{_}^{hi} clones. C1, E9, and C7 clones are evaluated in the 12^{th}, 20^{th}, and 50^{th} passages. CAR⁺ denotes CAR⁺/mPSCs. H denotes heat inactivation.

FIG. 7 shows tumorigenic capacity of CAR⁺/mPSCs^{Oct-4}^{_}^{hi} C1 clone. (A) Tumor formation of C1 clone is performed on SCID mice. C1 clone (n=6) or CAR⁺/mPSCs (n=4) in 1×10⁶ cells mixed in matrigel are subcutaneously transplanted on the back of SCID. Tumors growth is monitored by calipers gauge. (i), After 28 days, C1 clone derived tumors are observed, the arrow head indicates the C1 clone cell injection and the arrow indicates as CAR⁺/mPSCs injection. Tumors are excised for further inspection. (ii), Tumor growth curve are calculated based on the data collected on the following days after injection as indicated. Data are mean±SD of independent tumor measurement. (B) Representative H&E stained images of C1 clone-derived tumors. (i), Cells with a high nuclear/cytoplasmic ratio are shown. (ii), Magnified image of the boxed area in plane A-i. (iii), Tumor cells with a high mitotic rate are indicated with arrow heads. (iv), Magnified image of the boxed area in plane A-iii. (Scale bar: 100 μm)

FIG. 8 shows an immunohistochemical examination of C1 clone-derived tumors. Representative immunohistochemical images of C1 clone-derived tumors are shown. (A), Oct-4 and CAR expression. (B) Oncogenes activation, including phospho-Src, phospho-β-catenin, c-myc, and cyclin D1. (C) Expression of human adenocarcinoma diagnosis markers, including TTF1, NAPSA, CK7, and CK-HMW. Inserts are magnified images of boxed areas. (Scale bar: 100 μm)

FIG. 9 shows in vitro tumorigenic phenotype of CAR⁺/mPSCs^{Oct-4}^{_}^{hi}. (A) The soft agar colony formation assay. CAR⁺/mPSCs, CAR⁺/mPSCs^{Oct-4}^{_}^{hi} C1, E9, and C7 clones and A549 are subjected to soft agar culture. Colonies are photographed and quantified after 2 wk. (B) Sphere formation assay. CAR⁺/mPSCs. CAR⁺/mPSCs^{Oct-4}^{_}^{hi} C1, E9. and C7 clones and A549 are subjected to a sphere formation assay. Secondary spheres (>70 μm) are photographed and quantified after 10 days. (Scale bar: 100 μm)

FIG. 10 shows in vivo tumorigenic and metastatic capacities of CAR⁺/mPSCs^{Oct-4}^{_}^{hi} C1 clone. (A) In vivo xenograft tumor formation. (i), Growth curve of tumors. Different concentrations of the C1 clone (10⁵, 10⁴, 10³, and 10² cells) are subcutaneously injected into SCID mice. Tumor diameters are measured using calipers at the indicated times after injection. Data are presented as mean±SD. (ii), Tumors are excised, photographed, and measured at day 28 after injection. (Scale bar, 1 cm.) (B) Metastatic tumor nodule formation. The C1 clone (3×10⁵ cells) is injected into the tail vein of SCID mice. CAR⁺/mPSCs are injected as a native control. Metastatic tumor nodule formation in the lung is recorded after 5 wk (indicated by arrows). H&E staining of mice injected with the C1 clone shows extensive hemorrhage and nodule formation. The magnified image of C1 clone shows nodules in lung tissue. (Scale bar, 100 μm.) (C) Kaplan-Meier survival curves of CAR⁺/mPSCs- and C1 clone-injected mice (n=10).

FIG. 11 shows putative CICs traits in CAR⁺/mPSCs^{Oct-4}^{_}^{hi}. (A) Flow cytometry analysis of CD133 expression in CAR⁺/mPSCs and CAR⁺/mPSCs^{Oct-4}^{_}^{hi} C1, E9, and C7 clones. Percentages indicate the CD133-positive population for each clone. (B) Flow cytometry analysis of ALDH activity. The results of the ALDEFLUOR assay with CAR⁺/mPSCs and CAR⁺/mPSCs^{Oct-4}^{_}^{hi} C1, E9, and C7 clones are shown. DEAB-treated samples serve as negative controls. Percentages indicate the ALDH-positive population for each clone. (C) Cell viability of CAR⁺/mPSCs^{Oct-4}^{_}^{hi}. CAR⁺/mPSCs^{Oct-4}^{_{—hi}} C1, E9, and C7 clones and A549 cells are treated with (i), cisplatin (2.5, 5, 10, 25, 50, and 100 μM) or (ii), paclitaxel (2.5, 5, 10, 50, 100, and 200 nM) for 48 h. Data are shown as mean±SD. *P<0.05, ** P<0.01 compared with A549 cells. (D) Anti-apoptosis potential of CAR⁺/mPSCs^{Oct-4}^{_}^{hi} clones. (i), Survivin expression in CAR⁺/mPSCs and CAR⁺/mPSCS^{Oct-4}^{_}^{hi} C1, E9, and C7 clones is analyzed using Western blot. (ii), Cleaved caspase-3 (c caspase-3) and cleaved caspase-9 (c caspase-9) levels in CAR⁺/mPSCs and CAR⁺mPSCs^{Oct-4}^{_}^{hi} C1, E9, and C7 clones after 10 μM cisplatin or 10 nM paclitaxel treatment.

FIG. 12 shows pro-angiogenic factors expression of CAR⁺/mPSCs^{Oct-4}^{_}^{hi}. (A) Gene expression levels of proangiogenic factors in CAR⁺/mPSCs and CAR⁺/mPSCS^{Oct-4}^{_}^{hi} C1, E9, and C7 clones are analyzed using real-time PCR. Data are presented as mean±SD. *P<0.05, ** P<0.01, compared with CAR⁺/mPSCs.

FIG. 13 shows angiogenic potential of CAR^{|}/mPSCs^{Oct-4 hi}. (A) CAM assay of CAR⁺/mPSCs and CAR⁺/mPSCs^{Oct-4}^{_}^{hi} C1, E9, and C7 clones. (i), Representative photomicrographs of cell transplantation after 72 h. Arrows indicate the branching points of blood vessels. (ii), Angiogenic potential is determined by counting the branch points. Matrigel alone is used to determine the background level and VEGF (10 ng) is used as a positive control. Data are expressed as mean±SD. #P<0.05 compared with Matrigel alone. **P<0.01 compared with CAR⁺/mPSCs. (B) Immunohistochemical staining of CD31 expression in CAR⁺/mPSCs^{Oct-4}^{_}^{hi} C1, E9, and C7 clones and A549 derived tumors. (i), Representative images of each tumor are shown. (Scale bar, 100 μm.) (ii), Quantification of CD31 expression in the tumors by TissueGnostics scanning and HistoQuest software analysis. Data are expressed as the mean±SD. **P<0.01 compared with A549 tumors.

FIG. 14 shows tube formation of CAR⁺/mPSCs^{Oct-4}^{_}^{hi} C1 clone co-cultured with SVEC4-10. Confocal microscopic images show the tube architecture of SVEC4-10 and C1 clone co-culture. SVEC4-10, mouse endothelial cell line, is stained in PKH26. C1 clone sphere or C1 clone cells are labeled with Calcein-AM. After 8 hours co-culture, tube construct are photographed. DAPI is used as nucleus marker. (A) C1 clone sphere co-cultured with SVEC4-10. (B) C1 clone cells co-cultured with SVEC4-10.

FIG. 15 shows tube formation assay for CAR⁺/mPSCs^{Oct-4}^{_}^{hi}. (i), After incubation, in EGM for 7 d, tube formation is detected in CAR⁺/mPSCs^{Oct-4}^{_}^{hi} C1, E9, and C7 clones. No tube formation is observed in EGM cultured CAR⁺/mPSCs. The tube network is stained using calcein-AM and recorded by fluorescence microscopy for 8 h. (ii), Tube formation capacity is determined by quantifying the tubular length. Data are expressed as the mean±SD. **P<0.01 compared with CAR⁺/mPSCs. (Scale bar: 100 μm)

FIG. 16 shows EGM cultured CAR⁺/mPSCs^{Oct-4}^{_}^{hi} showed the surface markers of endothelial cells. CAR⁺/mPSCs^{Oct-4}^{_}^{hi} C1, E9, and C7 clones are incubated in endothelial cells growth medium (EGM) for 7 days, and then analysed the expression of endothelial cells specific surface markers, including CD31, CD105, CD34, and CD144. The number indicates positive expression population.

FIG. 17 shows angiogenesis potential of CAR⁺/mPSCs^{Oct-4}^{_}^{hi}. (A) Immunofluorescence staining for the expression of endothelial antigens in C1-GFP clone-derived tumors. (i), CD31 is identified. (ii), vWF is identified. (iii) CD105 is identified. The magnified image shows that some GFP⁺ cells are involved in blood vessel formation (indicated by arrow), and a proportion of GFP⁺ cells also express CD31 (indicated by asterisk). (Scale bar: 100 μm.) (B) CD31 expression in dissociated tumors of the C1-GFP clone is analyzed using flow cytometry. CD31 sub-fraction, representing endothelial cells, constitute 3% of the whole tumor population. GFP expression is found in 18% of the CD31 endothelial cell sub-fraction.

FIG. 18 shows ANGs/Tie2 signaling analysis in EGM cultured CAR⁺/mPSCs^{Oct-4}^{_}^{hi}. (A) Real-time PCR is performed to analyze the gene expression of angiogenesis associated receptor, including VEGFR2 and Tie2, in EGM cultured CAR⁺/mPSCs^{Oct-4}^{_}^{hi} C1, E9. and C7 clones and CAR⁺/mPSCs. Data are presented as the mean±SD. **P<0.01 compared with CAR⁺/mPSCs. (B) Western blot is performed to analyze the ANGs/Tie2 signaling activation in EGM cultured CAR⁺/mPSCs^{Oct-4}^{_}^{hi} C1, E9, and C7 clones and CAR⁺/mPSCs, including Tie2, phospho-Tie2, ANG1 ANG2, GRB2, ERK and phospho-ERK expression.

FIG. 19 shows Tie2 kinase inhibitor reduces angiogenesis of CAR⁺/mPSCs^{Oct-4}^{_}^{hi}. (A) CAR^{|}/mPSCs^{Oct-4 hi} C1 clone is cultured in EGM medium for 7 days, and then tube formation assay is performed. Relative node number is used to validate the tube formation. Tube formation ability of EGM cultured C1 clone is reduced via 2 μM Tie2 kinase inhibitor treatment. Data are presented as the mean±SD. **P<0.01 compared with the group of without Tie2 kinase inhibitor. (B) CAM assay of C1 clone. Branch point number of blood vessel is used to validate the blood vessel induction of C1 clone. Tie2 kinase inhibitor (2 μM) decreases the blood vessel formation induced by C1 clone. Data are presented as the mean±SD. *P<0.05 compared with the group of without Tie2 kinase inhibitor. (C) in vivo xenograft tumor formation assay, the tumor growth of C1 clone is inhibited via Tie2 kinase inhibitor treatment. 50 mg/kg BW of Tie2 kinase is administrated by ip injection once every two days from day 7 to day 25. Tumor volume is recorded from 10 to 25 days. Data are presented as the mean±SD. *P<0.05 compared with the group of without Tie2 kinase inhibitor.

### SUMMARY OF THE INVENTION

The present invention relates to a cancer initiating cell comprising an isolated coxsackievirus and adenovirus receptor positive mouse pulmonary stem/progenitor cell that overexpresses Oct-4 (CAR⁺/mPSC^{Oct-4}^{_}^{hi}).

### DETAILED DESCRIPTION OF THE INVENTION

Solid tumors are thought to arise in organs that contain stem cell populations. The tumors in these organs consist of heterogeneous populations of cancer cells that differ markedly in their ability to proliferate and form new tumors; this difference in tumor-forming ability has been reported for example with breast cancer cells and with central nervous system tumors. While the majority of the cancer cells have a limited ability to divide, recent studies suggest that a population of cancer cells, termed cancer stem cells or cancer initiating cells (CICs), has the exclusive ability to extensively self-renew and form new tumors. Growing evidence suggests that pathways that regulate the self-renewal of normal stem cells are deregulated or altered in cancer stem cells, resulting in the continuous expansion of self-renewing cancer cells and tumor formation.

In this invention, cancer initiating cells (CICs) are generated in animal model to better understand the properties and characteristics of CICs, and these findings can aid cancer research to provide insight into early diagnosis and treatment of lung cancer. In previous studies, mouse pulmonary stem/progenitor cells (mPSCs) were enriched by using serum-free primary selection culture followed by FACS isolation using the coxsackievirus and adenovirus receptor (CAR) as the positive selection marker in the culture. These CAR⁺/mPSCs exhibited stem/progenitor properties, could differentiate into type-I pneumocytes, and possessed angiogenic potential. The present invention identifies pulmonary Oct-4+ stem/progenitor cells and demonstrates their susceptibility to SARS coronavirus (SARS-CoV) infection in vitro. Lung, stem/progenitor cells differentiate into alveolar pneumocytes and angiogenesis is induced within a 3D gelatin-microbubble scaffold. The present invention demonstrates that CAR⁺/mPSCs can be transformed via the overexpression of Oct-4 and then develop the typical CICs phenotype and type-I pneumocytes derived from CAR⁺/mPSCs are tested as well. In the experiments described herein, the characteristics of the transformed cells are examined using in vitro assays, including cell cycle and telomerase activity analysis, sphere forming assay, detection of CD133 expression and ALDH activity, and chemoresistance assay. In addition, in vivo assays, including limiting dilution transplantation and tumor metastasis assays in SCID mice, are used to further study the characteristics of the transformed cells. Since the capacity to induce angiogenesis is another trait of CICs, endothelial tube formation assay and in ovo chicken chorioallantoic membrane (CAM) assay are used to evaluate the angiogenic potential of the transformed cells.

In the present invention, overexpression of the pluripotent transcription factor Oct-4 in CAR⁺/mPSCs generated transformed cells is demonstrated, which is referred to as CAR⁺/mPSCs^{Oct-4}^{_}^{hi}. These transformed cells possess cancer/tumor initiating capacity and chemoresistance, as well as exhibiting remarkable expression of certain proangiogenic factors, including angiopoietins (ANGs) and VEGF, and enhanced angiogenic potential. Besides, the CAR⁺/mPSCs^{Oct-4}^{_}^{hi} exhibits the expression of the endothelial cells markers, including CD31, CD105, CD34, and CD144. Moreover, CAR⁺/mPSCs^{Oct-4}^{_}^{hi} actively participates in tumor blood vessel formation and activates the ANGs/Tie2 signaling pathway. These findings provide novel insights into the possible origin and generation of CICs, help elucidate the pathways responsible for CICs-mediated blood vessel formation, and offer new strategies for anti-angiogenic therapy in lung cancer.

As used herein, “a” or “an” may mean one or more. As used herein in the claim(s), when used in conjunction with the word “comprising”, the words “a” or “an” may mean one or more than one.

Therefore, the present invention provides a cancer initiating cell (CIC) which comprises an isolated coxsackievirus and adenovirus receptor positive mouse pulmonary stem/progenitor cell (CAR⁺/mPSC) that overexpresses Oct-4 (CAR⁺/mPSC^{Oct-4}^{_}^{hi}).

As used in the specification and claims, the term “cancer initiating cell (CIC)” is interchangeable and refers to a solid cancer stem cell. The cancer initiating cell is defined and functionally characterized as a small subset of cells from a tumor that can grow indefinitely in vitro under appropriate conditions (ability for self-renewal), is able to form tumors in vivo using only a small number of cells (<10² cells). Other common approaches to characterize CIC involve morphology and examination of cell surface markers, transcriptional profile, and drug response.

The CAR⁺/mPSC that overexpresses Oct-4 means the expression level of the Oct-4 in the CAR⁺/mPSC is 10 times higher than that of a normal cell. In a preferred embodiment, the expression level of the Oct-4 in the CAR⁺/mPSC is 16 times higher than that of a normal cell. In a more preferred embodiment, the expression level of the Oct-4 of the CAR⁺/mPSC is 20 times higher than that of a normal cell. As used herein, the normal cell is a normal CAR⁺/mPSC. The expression level is an expression level of a DNA, a RNA or a protein. In a preferred embodiment, the expression level is the expression level of the protein. The protein is encoded by an Oct-4 gene. In a more preferred embodiment, the sequence of the Oct-4 gene is SEQ ID NO: 1. In another embodiment, the Oct-4 gene is an Oct-4 cDNA.

In another embodiment, the CAR⁺/mPSC comprises a vector, wherein the vector comprises a nucleotide sequence for encoding an Oct-4 gene. In a preferred embodiment, the sequence of the Oct-4 gene is SEQ ID NO: 1.

In an embodiment, the CAR+/mPSC^{Oct-4}^{_}^{hi} exhibits soft agar colony formation, a sphere formation and an immortalization of characteristics.

In another embodiment, the CAR+/mPSC^{Oct-4}^{_}^{hi} has a cancer cell function, wherein the cancer cell function comprises cell proliferation, cell migration, cell invasion or combination thereof.

In an embodiment, the CAR+/mPSC^{Oct-4}^{_}^{hi} possesses a tumor initiating capacity. In a preferred embodiment, the CAR+/mPSC^{Oct-4}^{_}^{hi} has a tumorigenic capacity, wherein the tumorigenic capacity comprises tumor formation, tumor regeneration, metastatic capacity or combination thereof. Some embodiments of the CAR+/mPSC^{Oct-4}^{_}^{hi} of this invention grows indefinitely and forms tumors from <10³ cells in vitro. In a preferred embodiment, the CAR+/mPSC^{Oct-4}^{_}^{hi} grows indefinitely and forms tumors from <10² cells in vitro.

Different biomarkers for lung CICs have been proposed including CD133 expression, aldehyde dehydrogenase (ALDH) activity, and chemoresistance. In an embodiment, the CAR+/mPSC^{Oct-4}^{_}^{hi} exhibits CD133 expression, ALDH activity, chemoresistance or combination thereof. The chemoresistance means that the CAR+/mPSC^{Oct-4}^{_}^{hi} is resistant to a chemo-radiotherapy and/or a chemo-drug. Therefore, the cancer initiating cell is a lung cancer initiating cell. In an embodiment, the CAR+/mPSC^{Oct-4}^{_}^{hi} is a lung cancer initiating cell.

In one embodiment, the CAR+/mPSC^{Oct-4}^{_}^{hi} has a function for angiogenesis. In a preferred embodiment, the CAR+/mPSC^{Oct-4}^{_}^{hi} has a function for participating in a blood vessel formation. Therefore, the CAR+/mPSC^{Oct-4}^{_}^{hi} not only possesses angiogenic potential but also participates in the tumor blood vessel formation. In another embodiment, the CAR+/mPSC^{Oct-4}^{_}^{hi} has a function for ANG/Tie2 signal pathway to enhance the angiogenesis. In a preferred embodiment, the CAR+/mPSC^{Oct-4}^{_}^{hi} has a function for activating Tie2 signal pathway to enhance the angiogenesis.

In another embodiment, the CAR+/mPSC^{Oct-4}^{_}^{hi} expresses a surface marker of an endothelial cell, wherein the surface marker of the endothelial cell comprises CD31, CD105, CD34, CD144 or combination thereof.

The present invention also provides an use of a animal with a tumor for screening an anti-cancer drug, wherein the tumor is induced by a cancer initating cell (CIC), wherein the cancer initiating cell comprises an isolated coxsackievirus and adenovirus receptor positive mouse pulmonary stem/progenitor cell (CAR⁺mPSC) that overexpress Oct-4 (CAR⁺/mPSC^{Oct-4}^{_}^{hi}).

In one embodiment, the animal is a mouse.

The phrases “isolated” refer to material, which is substantially or essentially free from components which normally accompany it as found in its native state.

“Anti-cancer drug” refers to a drug comprising a composition having an anti-tumor activity as its active ingredient. “Anti-tumor activity” refers to a tumor growth suppressing effect, a tumor cytotoxic effect and/or a tumor-regression effect.

The CAR⁺/mPSC that overexpresses Oct-4 means the expression level of the Oct-4 in the CAR⁺/mPSC is 10 times higher than that of a normal cell. In a preferred embodiment, the expression level of the Oct-4 in the CAR⁺/mPSC is 16 times higher than that of a normal cell. In a more preferred embodiment, the expression level of the Oct-4 of the CAR⁺/mPSC is 20 times higher than that of a normal cell. As used herein, the normal cell is a normal CAR⁺/mPSC. The expression level is an expression level of a DNA, a RNA or a protein. In a preferred embodiment, the expression level is the expression level of the protein. The protein is encoded by an Oct-4 gene. In a more preferred embodiment, the sequence of the Oct-4 gene is SEQ ID NO: 1.

In another embodiment, the CAR^{|}/mPSC comprises a vector, wherein the vector comprises a nucleotide sequence for encoding an Oct-4 gene. In a preferred embodiment, the sequence of the Oct-4 gene is SEQ ID NO: 1.

In one embodiment, the cancer initiating cell is a lung cancer initiating cell. In a preferred embodiment, the tumor is a lung tumor. In another embodiment, the anti-cancer drug is a drug for treating a cancer initiating cell. In a preferred embodiment, the anti-cancer drug is an anti-lung cancer drug. In a more preferred embodiment, the anti-cancel drug is a drug for treating a lung cancer initiating cell.

The term “anti-cancer” as described herein comprises treating cancer and inhibiting cancer. Moreover, the “anti-cancer” comprises treating and/or inhibiting the cancer initiating cell. As used herein, the term “treating” comprising curing, healing, alleviating, relieving, altering, remedying, ameliorating, improving or affecting the disease, the symptoms of disease, or the predisposition toward disease. Treating and/or inhibiting the cancer initiating cell can include, for example, ameliorating, preventing, eliminating, or reducing the number of CICs in a subject, eliminating CICs in a subject, etc.

In one embodiment, the subject is an animal. Preferably, the subject is a mammal. More preferably, the subject is a human.

The present invention further provides a method for screening an anti-cancer drug, comprising: (a) implanting cancer initiating cells (CICs) into an animal, wherein the cancer initiating cells comprise isolated coxsackievirus and adenovirus receptor positive mouse pulmonary stem/progenitor cells (CAR^{|}/mPSCs) that overexpress Oct-4 (CAR⁺/mPSC^{Oct-4}^{_}^{hi}), wherein the CICs develop and form a tumor; (b) administering a candidate drug to the animal; and (c) evaluating an effect of the candidate drug on the tumor containing the CICs.

In one embodiment, the anti-cancer drug is a drug for treating a cancer initiating cell. In a preferred embodiment, the anti-cancer drug is an anti-lung cancer drug. In a more preferred embodiment, the anti-cancer drug is a drug for treating a lung cancer initiating cell.

In another embodiment, the animal is a rodent, preferably a rat or a mouse.

The CAR^{|}/mPSCs that overexpress Oct-4 means the expression level of the Oct-4 gene in each CAR⁺/mPSCs is 10 times higher than that of a normal cell. In a preferred embodiment, the expression level of the Oct-4 gene in each CAR⁺/mPSCs is 16 times higher than that of a normal cell. In a more preferred embodiment, the expression level of the Oct-4 gene of each CAR⁺/mPSCs is 20 times higher than that of a normal cell. The expression level is an expression level of a DNA, a RNA or a protein. In a preferred embodiment, the expression level is the expression level of the protein. The protein is encoded by an Oct-4 gene. In a more preferred embodiment, the sequence of the Oct-4 gene is SEQ ID NO: 1. In another embodiment, the Oct-4 gene is a Oct-4 cDNA.

In another embodiment, each CAR⁺/mPSCs comprises a vector, wherein the vector comprises nucleotide sequence for encoding an Oct-4 gene. In a preferred embodiment, the sequence of the Oct-4 gene is SEQ ID NO: 1.

The term “tumor” refers to benign as well as to malignant neoplasias in their respective stages. The first stage of neoplastic progression is an increased number of relatively normal appearing cells, the hyperplastic stage. There are several stages of hyperplasia in which the cells progressively accumulate and begin to develop an abnormal appearance, which is the emergence of the dysplastic phase.

The term “candidate drug” as used herein, means any molecule, e.g. a protein or a pharmaceutical, i.e., a drug, with the capability of substantially inhibiting the growth of a tumor cell.

In an embodiment, the cancer initiating cells (CICs) are lung, cancer initiating, cells. In a preferred embodiment the tumor is a lung tumor. In the latter case initial evaluation of the effects of the candidate drug will be, e.g., the visual assessment of the size and severity of the tumor. This has the additional advantage that the visual inspection of the tumor allows an immediate and continuous assessment of drug efficacy. In the case of non visible tumors, drug effect evaluation will usually require the animal to be sacrificed to inspect the tumor. Neoplasias can be detected according to standard techniques well known to those of skill in the art. Such methods include, apart from visual inspection (for lesions on the skin), histochemical and immunohistochemical techniques, and the like. Typically the drug candidate(s) are evaluated for their ability to inhibit the formation and/or the growth of tumors developed from the transplanted cell line. The present invention further comprises a step of determining whether the candidate drug is the anti-cancer drug according to a result of the evaluating effect of the candidate drug from the step (c).

In addition, the present invention provides a method for screening an anti-cancer drug, comprising: (1) providing a tumor tissue, wherein the tumor tissue comprises cancer initiating cells (CICs), wherein the cancer initiating cells comprise isolated coxsackievirus and adenovirus receptor positive mouse pulmonary stem/progenitor cells (CAR⁺/mPSCs) that overexpress Oct-4 (CAR⁺/mPSC^{Oct-4}^{_}^{hi}); (2) contacting said tumor tissue with a candidate drug; and (3) detecting an effect of the candidate drug on the tumor tissue.

In one embodiment, the cancer initiating cells are a lung cancer initiating cell. In a preferred embodiment, the tumor tissue is a lung tumor tissue. In another embodiment, the anti-cancer drug is a drug for treating a cancer initiating cell. In a preferred embodiment, the anti-cancer drug is an anti-lung cancer drug. In a more preferred embodiment, the anti-cancer drug is a drug for treating a lung cancer initiating cell.

The CAR⁺/mPSC that overexpresses Oct-4 means the expression level of the Oct-4 in the CAR⁺/mPSC is 10 times higher than that of a normal cell. In a preferred embodiment, the expression level of the Oct-4 in the CAR⁺/mPSC is 16 times higher than that of a normal cell. In a more preferred embodiment, the expression level of the Oct-4 of the CAR⁺/mPSC is 20 times higher than that of a normal cell. The expression level is an expression level of a DNA, a RNA or a protein. In a preferred embodiment, the expression level is the expression level of the protein. The protein is encoded by an Oct-4 gene. In a more preferred embodiment, the sequence of the Oct-4 gene is SEQ ID NO: 1.

In another embodiment, each CAR⁺/mPSCs comprises a vector, wherein the vector comprises nucleotide sequence for encoding an Oct-4 gene. In a preferred embodiment, the sequence of the Oct-4 gene is SEQ ID NO: 1.

In one embodiment, the step of detecting the effect of the candidate drug on the tumor tissue comprises observing a change in the cancer initiating cell over time, cancer development process, or a biological property thereof, in the tumor tissue. The present invention further comprises a step of determining whether the candidate drug is the anti-cancer drug according to a result of the inhibiting effect of the test compound from the step (3).

The present invention also provides a method for scanning a candidate drug for anti-cancer, comprising: (i) collecting a culture solution containing cancer initiating cells (CICs), wherein the cancer initiating cells comprise isolated coxsackievirus and adenovirus receptor positive mouse pulmonary stem/progenitor cells (CAR⁺/mPSCs) that overexpress Oct-4 (CAR⁺/mPSC^{Oct-4}^{_}^{hi}); (ii) extracting an exosomal protein from CICS; (iii) analyzing the exosomal protein; and (iv) comparing a drug database with an analyzing result from step (iii) to obtain the candidate drug.

In one embodiment, the cancer initiating cells are a lung cancer initiating cell. In a preferred embodiment, the candidate drug for anti-cancer is a candidate drug for treating a cancer initiating cell. In a more preferred embodiment, the candidate drug for anti-cancer is a candidate drug for treating a lung cancer initiating cell. In another embodiment, the candidate drug for anti-cancer is a candidate drug for anti-lung cancer.

In another embodiment, the drug database is a DrugBank.

In one embodiment, the present invention further comprises a step after the step (iv), which is (v) assaying a cell toxicity of the candidate drug to CICs. If the candidate drug has a significant toxic effect on the cancer initiating cell, the candidate drug is an anti-cancer drug or a drug for treating cancer initiating cell.

Besides, the present invention provides a method of preparing a population of cancer initiating cells, comprising the steps: (1) providing vectors comprising a nucleotide sequence for encoding Oct-4 cDNA; (2) transfecting the vectors into a population of coxsackievirus and adenovirus receptor positive mouse pulmonary stem/progenitor cells (CAR+/mPSCs), wherein the vectors overexpress Oct-4 cDNA by increasing the number of copies of the nucleotide sequence in the CAR+/mPSCs relative to the number of copies that is normally present in a wild-type CAR+/mPSCs; and (3) isolating the population of CAR+/mPSCs that overexpress Oct-4 (CAR⁺/mPSC^{Oct-4}^{_}^{hi}) from the step (2).

In one embodiment, the gene sequence of the Oct-4 cDNA is SEQ ID NO: 1.

In another embodiment, the Oct-4 cDNA has a function for encoding an Oct-4 protein.

The term “vector” is used herein to refer to a nucleic acid molecule having nucleic sequences that enable its replication in a host cell. A vector can also include nucleic sequences to permit ligation of nucleic sequences within the vector, wherein such nucleic sequences are also replicated in a host cell. Representative vectors include plasmids, cosmids, and viral vectors. Preferably, the vectors are retroviral vectors.

### EXAMPLES

The examples below are non-limiting and are merely representative of various aspects and features of the present invention.

### Example 1

### Materials and Methods

The coxsackievirus and adenovirus receptor positive mouse pulmonary stem/progenitor cells (CAR⁺/mPSC) were isolated from primary cultures according to coxsackievirus and adenovirus receptor positive (CAR-positive) expression by fluorescence-activated cell sorting (FACS). CAR⁺/mPSCs and CAR⁺/mPSC-derived type-I pneumocytes were transfected with retroviral vector encoding Oct-4 (SEQ ID NO: 1). Oct-4 hyperexpression cells, CAR⁺/mPSCs^{Oct-4}^{_}^{hi} clones were subjected to Western blot analysis, telomerase repeat amplification assay, flow cytometry analysis, soft agar colony formation assay, sphere formation assay, reverse transcription PCR and real-time PCR analysis gene expression, tube formation assay and CAM assay. In vivo tumorigenic potential was evaluated by limiting dilution transplantation and metastasis assays in SCID mice. Derived tumors were treated with immunohistochemical and immunofluorescence staining.

### Cell Culture

Human lung adenocarcinoma cell line A549, mouse axillary lymph node/vascular epithelial cell line SVEC4-10 and human embryonic kidney cell line (HEK) 293T were obtained from the Bioresource Collection and Research Center of Taiwan. A549, SVEC4-10 and HEK293T cells were maintained in Dulbecco's modified Eagle medium (DMEM, Sigma-Aldrich) with 10% FBS at 37° C. in humidified incubator with 5% CO₂.

### Serum-Free Primary Selection Culture of Mouse Pulmonary Stem/Progenitor Cells

Neonatal ICR mice (postnatal between 1 to 3 days) were sacrificed by cervical dislocation. The lung tissues were separated and collected in pre-chilled Hank's buffer with penicillin (100 units/mL) and streptomycin (100 g/mL). Lung tissues were cut into small pieces of 1 to 2 mm in diameter in digested medium containing 0.1% protease type-XIV (Sigma-Aldrich) and 1 ng/mL DNase-I (Sigma-Aldrich) in Minimum Essential Medium Eagle (MEM) medium at 4° C. overnight. Afterwards, 10% FBS/MEM medium was added to neutralize the protease/DNase-I and tissue suspensions were gently pipetted with 10-mL pipettes several times. Tissue debris was filtered through a 100 m nylon cell strainer. The cells were washed and resuspended in MCDB-201 medium (Sigma-Aldrich) supplemented with insulin/transferrin/selenium (ITS) (Invitrogen). These cells were cultivated at a density of 3 10⁵ cells/mL in collagen-I (Becton Dickinson Biosciences) coated cell culture dishes. After 1 day of incubation, the cells were refreshed on MCDB-201 medium supplemented with ITS and recombinant 1 ng/mL epidermal growth factors (Invitrogen). Pulmonary epithelial colonies formed in the culture when cells were confluent at day 10 to 14. These primary cells were applied to CAR-positive mPSCs isolation using FACS.

### CAR⁺/mPSCs Isolation

Cell suspensions obtained from the primary cultures were analyzed for CAR-positive cells using a FACS caliber instrument (Becton Dickinson Biosciences). Briefly, 1×10⁶ cells were incubated with goat polyclonal anti-CAR antibody (R&D Systems) at 4° C. for 1 h. After washing, cells were incubated with Alexa488-coupled donkey anti-goat IgG (Jackson ImmunoResearch) at 4° C. for 1 h. Cell fluorescence was evaluated using an FACSAria™ cell sorter (Becton Dickinson Biosciences), and data were analyzed using CellQuest™ (Becton Dickinson Biosciences). Cells were purified to >90% according to CAR-positive expression, and referred to as CAR⁺/mPSCs. CAR⁺/mPSCs were centrifuged using low speed centrifugation (1100 rpm for 5 min) and re-suspended for later use, including Oct-4 transfection and cell differentiation experiments.

### Oct-4 Transfection

CAR⁺/mPSCs were isolated from primary cultures according to CAR-positive expression by FACS as described previously. Detailed methods are described in Supplementary Methods. CAR⁺/mPSCs and CAR⁺/mPSCs-derived type-I pneumocytes were transfected with retroviral vectors encoding Oct-4 (SEQ ID NO: 1). Briefly, the retroviral vector plasmid pMXs-mOct-4 (Addgene) and packaging plasmids (pCMV-gag-pol-PA and pCMV-VSVg) were introduced into HEK293T cells using GeneJuice transfection reagent (Novagen). After 48 h, viral supernatants were passed through a 0.45 μm filter and supplemented with 10 μg/mL polybrene. CAR⁺/mPSCs and derived type-I pneumocytes were seeded at 1×10⁴ cells per 35 mm dish, and incubated in the viral supernatants for 16 h. Transfected cells were cultivated in mES/MCDB201 (1:1) medium and supplied with mitomycin C inactivated MEF cells (feeder cells). Cobblestone-like colonies formed between day 18 and day 25. At day 28, colonies were manually isolated and further expanded on Matrigel (Becton Dickinson Biosciences) supplement in mES/MCDB201 (1:1) medium to establish the C1, E9, and C7 cell clones. For C1-GFP clone generation, C1 clone was transfected with retroviral vectors encoding GFP. Briefly, the retroviral vector plasmid pMXs-puro GFP (Addgene) and packaging plasmids (pCMV-gag-pol-PA and pCMV-VSVg) were introduced into HEK293T cells using GeneJuice transfection reagent (Novagen). After 48 h, viral supernatants were passed through a 0.45 μm filter and supplemented with 10 μg/mL polybrene. C1 clone were seeded at 1×10⁴ cells per 35 mm dish and incubated in the viral supernatants for 16 h. Puromycin (2.5 μg/mL) was add to the medium, after 5 d, GFP-positive colonies were determined for expansion, referred to as C1-GFP clone.

### RNA Extraction, Reverse Transcription PCR, and Real-Time PCR

Total RNA was extracted using TRIzol (Invitrogen). For cDNA synthesis, M-MLV RT (Promega) was used according to the manufacturer's instructions. Reverse transcription PCR was performed using Taq polymerase (Invitrogen) according to the manufacture's protocol. Real-time PCR was performed using the 7900 HT real-time PCR instrument (Applied Biosystems). Primer sequences are listed in Table 1. Glyceraldehyde 3-phosphate dehydrogenase (GAPDH) expression was used for normalization.

TABLE 1
RT-PCR and Real-time primer sequence

| | | | | | |
|---|---|---|---|---|---|
| | | | | Anneal Temperature | Product Size |
| Gene | Accession | Forward Prmiers (5′→3′) | Reverse Primers (5′→3′ | (° C.) | (bp) |

RT-PCR

| | | | | | |
|---|---|---|---|---|---|
| CAR | NM_009988 | CGATGTCAAGTCTGGCGA | GAACCGTGCAGCTGTATG | 57 | 356 |
| Oct-4 | NM_013633 | ATGGCTGGACACCTGGCTTC | CCAGGTTCTCTTGTCTACCTC | 62 | 1121 |
| Sox2 | NM_011443 | TAGAGCTAGACTCGGGGCGATGA | TTGCCTTAAACAAGACCA | 562 | 297 |
| Nanog | NM_028016 | AAAGGATGAAGTGCAAGCGGTGG | CTGGCTTTGCCCTGACTTTAAGC | 58 | 520 |
| GAPDH | NM_008084 | ACCACAGTCCATGCCATCAC | TCCACCACCCTGTTGCTGTA | 58 | 452 |

Real-time PCR

| | | | |
|---|---|---|---|
| ANG1 | NM_009640 | GCATTCTTCGCTGCCATTCT | TCTCCCTCCGTTTTCTGGATT |
| ANG2 | NM_007426 | CCAACGCCTTAACCGATCTC | ACCCCGAGTCTGTGGATTGAC |
| VEGFα | NM_009595 | TTGTGTTGGGAGGAGGATGTC | GAAGCCTTTCATCCCATTGTCT |
| PLGH | NM_08827 | TGGCTGCTGTGGTGATGAA | TGCATAGTGATGTTGGCTGTCTT |
| PDGFα | NM_011057 | TTTCCAGACTTGGGCTTGGA | AACGGACCCCCAGATCAGA |
| GCSF | NM_009971 | GCAGGCTCTATCGGGTATTTCC | AGTTGGCAACATCCAGCTGAA |
| VCAM1 | NM_0011693 | TGCGAGTCACCATTGTTCTGAT | ACCCCTCCGTCCTCACCTT |
| bFGF | NM_008006 | TGGTATGTGCCACTGAAAGGA | TCCAGGTCCCGTTTTGGAT |
| VEGFR2 | NM_010612 | ACTGCAGTGATTGCCATGTTCT | TCATTGGCCCGCTTAACG |
| Tie2 | NM_013690 | CTTCATGTACAAGGGGCATTTC | GTGGGTGGCTTGCTTGGT |
| GAPDH | NM_008084 | CCAGCCTCGTCCCGTAGA | CGCCCAATACGGCCAAA |

### Western Blot Analysis

Cell lysates were extracted in RIPA buffer (Pierce) and quantified by a BCA protein assay kit (Pierce) according to the manufacturer's protocol. Equal amounts (30 μg) of total protein were separated using sodium dodecyl sulfate polyacrylamide gel electrophoresis and blotted onto activated polyvinylidene difluoride membranes (Millipore). After blocking with 5% fat-free milk, the membranes were incubated with primary antibodies, as listed in Table 2. The blots were then incubated with secondary antibody conjugated with horseradish peroxidase and immunoreacted bands were detected by enhanced chemiluminescence detection (Millipore).

### Flow Cytometry Analysis

In CD133 expression analysis, cells were dissociated into single cells, washed, and suspended in PBS. Cells were labeled with allophycocyanin (APC)-conjugated anti-mouse CD1133 (BioLegend), and then analyzed using the FACS caliber instrument. In cell cycle distribution analysis, cells were cultivated in 6-well plates. After incubating for 24 h, cells were collected, washed with PBS, and fixed in 70% ethanol at −20° C. overnight. Subsequently, the cells were washed once with PBS and re-suspended in PBS containing 200 μg/mL RNase A and 50 μg/mL propidium iodide. FACS caliber instrument was used to analyze the cell cycle distribution. CD31 and GFP expression in tumors, tissue dissociation kit (Miltenyi Biotec) was used to dissociate tumors into cell suspension. Cell suspension was stained with APC conjugated anti-mouse CD31 (BioLegend) and subsequently analyzed using the FACS-caliber instrument.

### ALDH Activity Assay

The aldehyde dehydrogenase (ALDH) activity of cells was detected using the ALDEFLUOR assay kit (StemCell Technologies) according to the manufacturer's protocol. The cells were suspended in an ALDEFLUOR assay buffer containing BODIPY-aminoacetaldehyde (BAAA) and incubated for 60 min at 37° C. The cells were treated with an ALDH inhibitor, diethylaminobenzaldehyde (DEAB), as a negative control. Propidium iodide staining identified nonviable cells. The FACS-caliber instrument was used to analyze the ALDH activity of cells in a green fluorescence channel (520-540 nm).

### Telomerase Repeat Amplification Assay

Telomerase activity was measured using the telomerase repeat amplification (TRAP) assay. Cells were homogenized in a TRAP lysis buffer. Protein (20 μg) was used in the telomerase reaction, along with 50 μL of a TRAP reaction buffer containing 20 mM Tris-HCl (pH 8.3), 1.5 mM MgCl₂, 63 mM KCl, 0.05% Tween-20, 1 mM EGTA, 50 μM deoxynucleotide triphosphate (Pharmacia), 0.1 μg each of labeled TS, ACX, and U2 primers, 5×10⁻³ attomoles of an internal control primer (TSU2), 2 units of Taq DNA polymerase (Invitrogen), and 2 μL of CHAPS extract. After incubating at 3° C. for 30 min, the telomerase-extended products were amplified through PCR under the following conditions: 30 cycles with each cycle comprising incubations at 94° C. for 30 s, 60° C. for 30 s, and 72° C., for 45 s. The reaction mixture was heated to 94° C. for 5 min to inactivate telomerase. Amplified products were resolved on a 12% polyacrylamide gel electrophoresis, stained with ethidium bromide and viewed under LTV light.

### Soft Agar Colony Formation Assay

A soft agar colony formation assay was performed by seeding 3×10³ cells in 35 mm tissue culture dishes containing a layer of 0.35% low-melting agarose/ES/MCDB-201 over a layer of 0.5% low-melting agarose/ES/MCDB-201. Additional complete media was added every 2 d. After 2 wk, colonies were fixed with 0.05% crystal violet and methanol and colony formation was photographed and quantified using light microscopy.

### Sphere Formation Assay

Cells were seeded in a 24 well ultra low-attachment plate (Corning) at a density of 1,000 cells per well and grown in serum-free DMEM, supplemented with 2% B27 (Invitrogen), 20 ng/mL EGF, and 20 ng/mL bFGF (Invitrogen). After cultivation for 14 d, primary spheres were harvested using centrifugation, dissociated with trypsin, and re-suspended in this medium. The secondary spheres (>70 μm) were photographed and quantified after 10 days.

### Xenograft Tumor Assay

All animal experiments were reviewed and approved by the Institutional Animal Care and Use Committee of National Taiwan University College of Medicine. For teratoma formation assay, 1×10⁶ cells of CAR⁺/mPSCs^{Oct-4}^{_}^{hi} C1, E9, and C7 clones were subcutaneously injected into 8-week-old male severe combined immunodeficiency (SCID) mice. For limiting dilution transplantation experiment, the C1 clone (10⁵, 10⁴, 10³ and 10² cells) or CAR⁺/mPSCs (10⁶ cells) were subcutaneously injected into SCID mice. For C1-GFP clone and A549 cells derived tumor experiments. C1-GFP clone (1×10⁵ cells) or A549 (1×10⁶ cells) were subcutaneously injected into SCID mice. Tumor dimensions were measured using calipers once every 3 d, and volumes (cm³) were calculated according to the standard formula: length×width²/2. At the end of the experiment, the tumors were surgically excised and photographed. In metastasis assay, 3×10⁵ cells of C1 clone, and CAR⁺/mPSCs were injected into the lateral tail vein of SCID mice. Lung metastatic nodules were evaluated at week 5 by necropsy and histological examination. Kaplan-Meier analysis was used for comparing the survival rates of mice injected with C1 clone and those injected with CAR⁺/mPSCs.

In secondary tumor experiments, 1×10⁶ of C1 clone developed tumor tissue after 4 weeks subcutaneous transplantation. Tumor tissue was cut into mini pieces and digested in trypsin-EDTA. Cell suspension was collected through 100 μm cellular strainer. Cell differentiated high and low CAR expression using FACSAriaII cell sorter. 1×10⁵, 10⁴, 10³ and 1×10² cell number (n=4/group) of CAR^{high} and CAR^{low} population were subcutaneously transplanted on the back of SCID mice. The secondary tumor formation was recorded for 5 weeks and then CAR expression in secondary tumor was further validated.

### Immunohistochemistry and Immunofluorescence Staining

Tumors were fixed in formalin and subsequently dehydrated, paraffin embedded, and sectioned. Tumor sections were subjected to antigen retrieval with microwave irradiation in a citrate buffer (10 nM, pH 6.0). The sections were incubated at 4° C. with primary antibody overnight. For immunohistochemical staining, the sections were incubated with corresponding HRP-coupling secondary antibodies at room temperature for 1 h, and visualized using 0.05% 3,3′-diaminobenzidine (DAB), and the nuclei were counter-stained with hematoxylin. For immunofluorescence staining, corresponding fluorescence coupling with a secondary antibody was performed at room temperature for 1 h. The nuclei were counter=stained with DAPI. Negative controls were prepared using identical conditions, and control IgG was used as a substitute for the primary antibody. Antibodies are listed in Table 2. Sections were examined using the Nikon Eclipse 800. Immunohistochemical staining sections were quantified using TissueFax (TissueGnostics GmbH) scanning, and the percentage of immune-positive population were analyzed with HistoQuest software (TissueGnostics GmbH).

TABLE 2
Antibody application

| Protein | Assay | Cat. No. | Company | Host | Dilution | Incubation Time |
|---|---|---|---|---|---|---|
| CAR | IF | AF2654 | R&D Systems | goat | 1:100 | O/N, 4° C. |
| T1α | IF | sc23564 | Santa Cruz | goat | 1:200 | O/N, 4° C. |
| AQP5 | IF | AB15858 | Millipore | rabbit | 1:200 | O/N, 4° C. |
| CAR | IHC | AF2654 | R&D Systems | goat | 1:100 | O/N, 4° C. |
| Oct-4 | IHC | sc5279 | Santa Cruz | mouse | 1:100 | O/N, 4° C. |
| phospho-Src | IHC | ab79308 | Abcam | rabbit | 1:100 | O/N, 4° C. |
| phospho-β-catemin | IHC | ab53050 | Abcam | rabbit | 1:100 | O/N, 4° C. |
| c-myc | IHC | ab32072 | Abcam | rabbit | 1:500 | O/N, 4° C. |
| cycline D1 | IHC | ab134175 | Abcam | rabbit | 1:200 | O/N, 4° C. |
| TTF1 | IHC | M3575 | Dako | mouse | 1:100 | O/N, 4° C. |
| NAPSA | IHC | NB110-68133H | Novus Biologicals | mouse | 1:500 | O/N, 4° C. |
| CK7 | IHC | ab9021 | Abcam | mouse | 1:1000 | O/N, 4° C. |
| CK-HMW | IHC | ab76714 | Abcam | mouse | 1:50 | O/N, 4° C. |
| CD31 | IHC | ab28364 | Abcam | rabbit | 1:100 | O/N, 4° C. |
| CD105 | IHC | ab107595 | Abcam | rabbit | 1:50 | O/N, 4° C. |
| vWF | IHC | ab9378 | Abcam | rabbit | 1:100 | O/N, 4° C. |
| Oct-4 | WB | sc5279 | Santa Cruz | mouse | 1:200 | O/N, 4° C. |
| Survivin | WB | ab182132 | Abcam | rabbit | 1:1000 | O/N, 4° C. |
| cleaved caspase-3 | WB | 9664 | Cell signaling | rabbit | 1:1000 | O/N, 4° C. |
| cleaved caspase-9 | WB | 9509 | Cell signaling | rabbit | 1:1000 | O/N, 4° C. |
| Tic2 | WB | sc9026 | Santa Cruz | rabbit | 1:200 | O/N, 4° C. |
| phospho-Tic2 | WB | ABS219 | Millipore | rabbit | 1:2000 | O/N, 4° C. |
| ANG1 | WB | sc6320 | Santa Cruz | goat | 1:200 | O/N, 4° C. |
| ANG2 | WB | 2948 | Cell signaling | rabbit | 1:1500 | O/N, 4° C. |
| Grb2 | WB | ab32037 | Abcam | rabbit | 1:1000 | O/N, 4° C. |
| ERK | WB | sc93 | Santa Cruz | rabbit | 1:500 | O/N, 4° C. |
| phospho-ERK | WB | sc7383 | Santa Cruz | mouse | 1:500 | O/N, 4° C. |
| GAPDH | WB | ab181602 | Abcam | rabbit | 1:1000 | O/N, 4° C. |
| CAR | FC | AF2654 | R&D Systems | goat | 1:100 | 1 hr, 4° C. |
| CD31-APC | FC | 102509 | BioLegand | | 1:100 | 1 hr, 4° C. |
| CD133-APC | FC | 141208 | BioLegand | | 1:100 | 1 hr, 4° C. |
| Control IgG | IF | 012-000-003 | Jackson ImmunoResearch | rat | 1:200 | O/N, 4° C. |
| Control IgG | IF | 005-000-003 | Jackson ImmunoResearch | goat | 1:200 | O/N, 4° C. |
| Control IgG | IF | 011-000-003 | Jackson ImmunoResearch | rabbit | 1:200 | O/N, 4° C. |
| Control IgG | FC | 400511 | BioLegand | | 1:100 | 1 hr, 4° C. |
| AlexaFlour ®488-anti-goat IgG | IF | 705-545-003 | Jackson ImmunoResearch | donkey | 1:200 | 1 hr, RT |
| Cy ™3-anti-goat IgG | IF | 705-165-147 | Jackson ImmunoResearch | donkey | 1:500 | 1 hr, RT |
| Cy ™3-anti-mouse IgG | IF | 115-165-003 | Jackson ImmunoResearch | goat | 1:500 | 1 hr, RT |
| Cy ™3-anti-rabbit IgI | IF | 711-165-152 | Jackson ImmunoResearch | donkey | 1:500 | 1 hr, RT |
| AlexaFlour ®488-anti-rabbit IgG | IF | 111-095-003 | Jackson ImmunoResearch | goat | 1:500 | 1 hr, RT |

IF, Immunofluorescence;
IHC, Immunohistochemistry;
WB, Western blot;
FC, Flow cytometry;
O/N, Overnight;
RT, Room temperature.

### Cell Viability Assay

Cells were seeded at 3×10³ cells per well in 96-well plates and incubated for 18 h. Cells were treated with cisplatin (Sigma-Aldrich) or paclitaxel (Sigma-Aldrich) at various concentrations. After 48 h, WST-1 assay (Roche) was performed to determine cell viability according to the manufacturer's instructions. Cell viability was expressed as a percentage of the non-treated group, and the IC₅₀ values were determined.

### Chick Chorioallantoic Membrane (CAM) Assay

Fertilized chicken eggs were incubated at 37° C. in an atmosphere of 80% humidity. At day 8 of the development, 1×10⁶ cells were loaded onto a membrane and implanted on the top of the growing CAM. At day 11, CAM was fixed with 4% paraformaldehyde, and photographed using a stereomicroscope and digital camera. Branching points were quantified using NIH Image J software with the angiogenesis plugin.

In Vitro CAR⁺/mPSCs^{Oct-4}^{_}^{hi} Tube Formation Assay

Cells were cultivated in an endothelial cell growth medium (EGM) (Lonza) for 7 d. Cells were collected and suspended in DMEM supplemented with 2% FBS and seeded on Matrigel. After 8 h, cells were stained with calcein-AM (Invitrogen), and images were obtained using a fluorescence microscope (Zeiss).

CAR⁺/mPSCs^{Oct-4}^{_}^{hi} C1 Clone and SVEC4-10 Co-Culture for Tube Formation

Matrigel was plated on 35 mm Ibidi μ dishes. C1 clone derived spheres labeled with the green fluorescent tracer, calcein-AM, were mixed with SVEC4-10 cells that had been stained with a red fluorescent cell tracer dye, PHK26 (Sigma-Aldrich). Nuclei were counter-stained with Hoechst33342. The cellular mixture was seeded onto Matrigel plated dishes in DMEM containing 2% FBS and 5% Matrigel. Tube formation was recorded using time-lapse immunofluorescence confocal microscopy.

### Inhibition of Tie2 Kinase Inhibitor

Tie2 kinase inhibitor is a potent, reversible and selective ATP-binding site-targeting Tie2 kinase. The chemical formula was 4-(6-methoxy-2-naphthyl)-2-(4-methylsulfinyl phenyl-5-(4-pyridyl)-1H-imidazole (CAS number:948557-43-5) (ab141270, Abcam, Cambridge, Mass.). The cytotoxicity of Tie2 kinase inhibitor (2 μM) for CAR⁺/mPSCs^{Oct-4}^{_{—hi}} clones were 93.2±2.51% of survival rate after 24 hours treatment.

In tube formation experiments, tube formation of EGM cultured CAR⁺/mPSCs^{Oct-4}^{_}^{hi} C1 clone was treated with 2 μM Tie2 kinase inhibitor. After 8 h, cells were stained with calcein-AM (Invitrogen), and images were obtained using a fluorescence microscope (Zeiss).

In blood vessel formation assay, 1×10⁶ cells of CAR⁺/mPSCs^{Oct-4}^{_}^{hi} C1 clone were treated with 2 μM Tie2 kinase inhibitor and proceeded CAM assay. At day 11, CAM was fixed with 4% paraformaldehyde, and photographed using a stereomicroscope and a digital camera. Branching points were quantified using NIH Image J software with the angiogenesis plugin.

1×10⁵ cells of CAR⁺/mPSCs^{Oct-4}^{_}^{hi} C1 clone were subcutaneously injected into 8-week-old male severe combined immunodeficiency (SCID) mice. 50 mg/kg BW Tie2 kinase inhibitor were administered via ip once every two days from 10 to 25 days. Tumor dimensions were measured using calipers once every 2 or 3 d, and volumes (cm³) were calculated according to the standard formula: length×width²/2. At the end of the experiment, the tumors were surgically excised and photographed.

### Statistical Analysis

Quantitative data from at least three independent experiments are expressed as mean±standard deviation (SD). Student's t-tests were used to compare the differences between groups. Survival curves were obtained using the Kaplan-Meier analysis. P<0.05 is considered statistically significant.

### Results

### Transfection of Oct-4 for Hyperexpression in CAR⁺/mPSCs

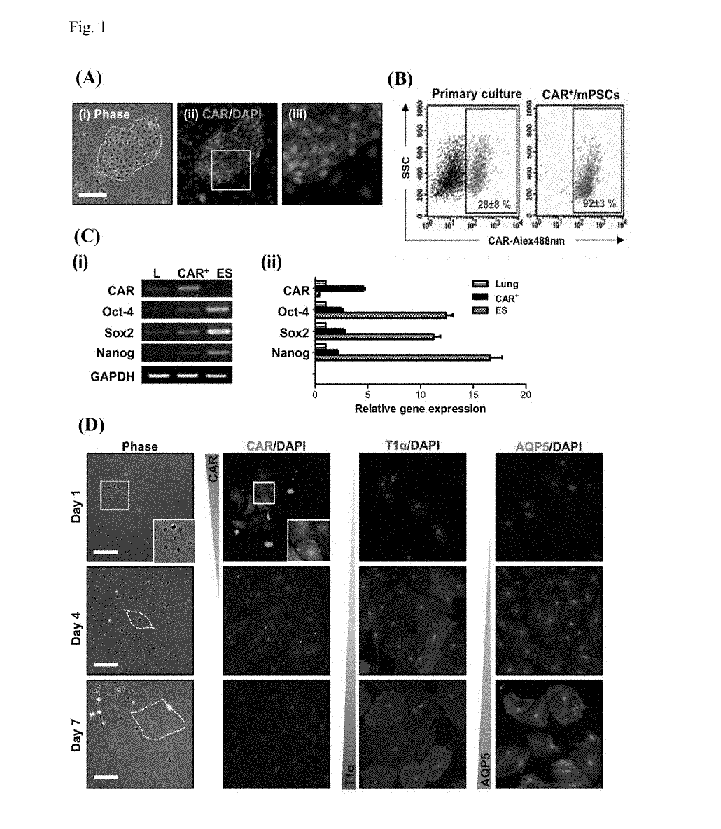

Tissue specific stem cells are small in number yet largely responsible for tissue homeostasis. In previous studies, CAR⁺/mPSCs were successfully identified and isolated (FIG. 1 (A) and (B)). Compared with the mouse embryonic stem cell line (E14), CAR^{|}/mPSCs had low expression levels of Oct-4, Sox-2 and Nanog in PCR and real-time PCR analysis (FIG. 1(C)). CAR⁺/mPSCs showed the potential to differentiate into type-I pneumocytes at day 7, evidenced by their flattened cellular morphology and by the presence of the type-I pneumocyte markers, T1α and AQP5 (FIG. 1(D)). Thus, CAR⁺/mPSCs possessed pulmonary specific stem/progenitor cell properties. These cells could be identified according to CAR expression and could be efficiently isolated using FACS.

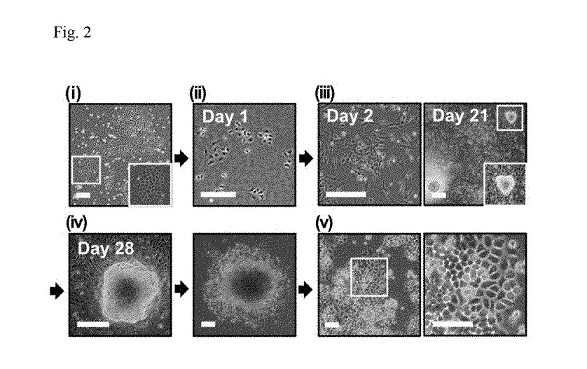

Overexpression of Oct-4 through retrovirus transfection was performed in both CAR⁺/mPSCs and CAR⁺/mPSCs-derived type-I pneumocytes. In the experiment, CAR⁺/mPSCs were transfected with Oct-4 (FIG. 2(i) and (ii)), feeder cells were supplied at day 2 (FIG. 2(iii)) and cobblestone-like colonies were first observed to form between day 18 and day 25. At day 28, the well-developed colonies exhibited phase-bright borders, and cells within the colonies had high nuclear/cytoplasmic ratios and prominent nucleoli (FIG. 2(iv)). The colonies were then picked and expanded to generate cell clones (FIG. 2(v)). In table 3, it showed frequency of cobblestone-like colony formation. CAR⁺/mPSCs with sham control transfection or in CAR⁺/mPSCs-derived type-I pneumocytes transfected with Oct-4 showed no detectable (N.D.) colony formation.

TABLE 3
Frequency of cobblestone-like colony formation

| | Frequency of |
|---|---|
| Cell Type | colony formation |
| CAR⁺/mPSCs | 0.09 ± 0.04% |
| CAR⁺/mPSCs with | N.D. |
| sham control | |
| CAR⁺/mPSCs-derived | N.D. |
| Type-I pneumocytes | |

Data are presented as mean ± SD

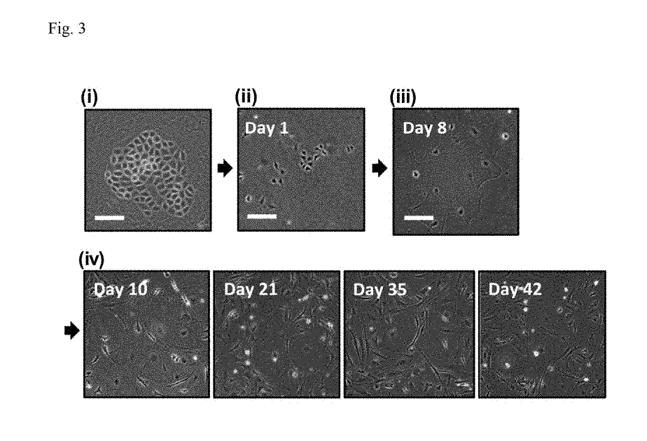

The frequency of cobblestone-like colony formation in CAR⁺/mPSCs ranged from 0.05-0.13% (Table 3). Meanwhile, no cobblestone-like colonies were observed in the sham control transfection in CAR⁺/mPSCs (data not shown). Retroviral transfection of CAR⁺/mPSCs-derived type-I pneumocytes was performed at day 8 when type-I pneumocytes were well-differentiated (FIG. 3(i), (ii) and iii), and feeder cells were supplied at day 10 (FIG. 3(iv)). Oct-4 transfected type-I pneumocytes had no detectable colony formation until day 42 after induction. These results indicated that overexpression of Oct-4 could induce cobblestone-like colony formation in CAR^{|}/mPSCs; and that cell status, stem/progenitor stage rather than differentiation stage, appear to be critical for the induction of colonies via Oct-4 overexpression.

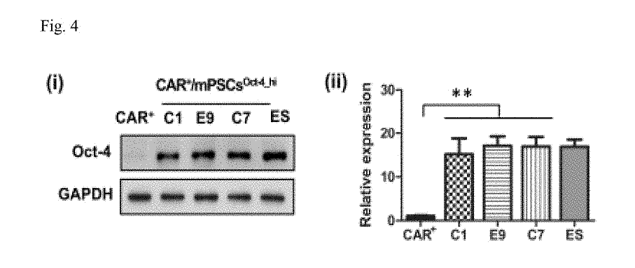

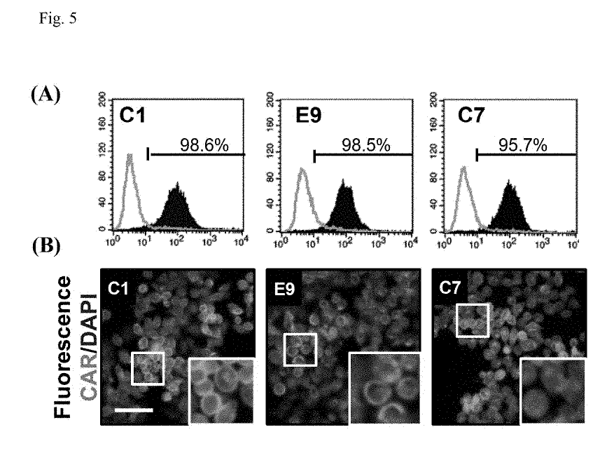

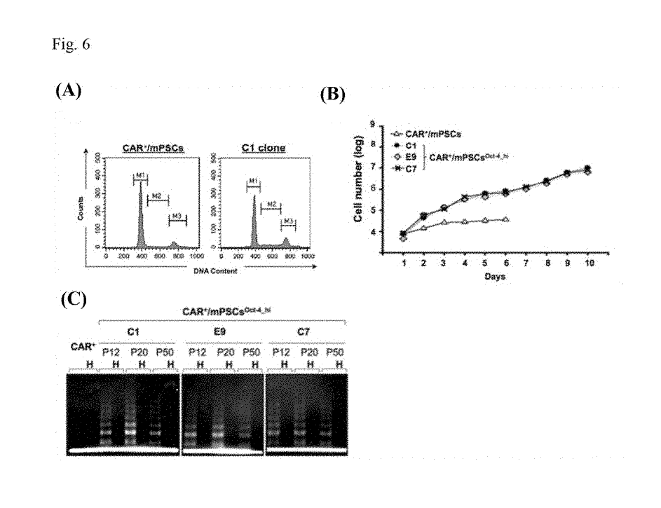

The cobblestone-like colonies were isolated and established as separate cell clones. To evaluate phenotypic alterations, cell clones from 3 independent experiments, named C1, E9, and C7, were selected for further examinations. Western blot analysis showed that Oct-4 was highly expressed in the C1, E9, and C7 clones; thus, they were referred as CAR⁺/mPSCs^{Oct-4}^{_}^{hi} clones (FIG. 4(i)). The expression level of the Oct-4 in the C C1, E9, and C7 clones was 16˜20 times higher than that of the CAR⁺/mPSC. The Oct-4 expression levels of C1, E9, and C7 clones were similar to that of the mouse embryonic stem cell line (E14), whereas CAR⁺/mPSCs exhibited low Oct-4 expression (FIG. 4(ii)). In primary cultures, CAR was specifically expressed in pulmonary stem/progenitor cells and served as the marker for CAR^{|}/mPSCs isolation. In CAR⁺/mPSCs^{Oct-4}^{_}^{hi} clones, CAR was expressed in >95% of cells and these cells had lost the capacity to differentiate into type-I pneumocytes (FIG. 5(A) and (B)). Cell cycle analysis showed significant G₁-, S-, and G₂/M-phase shifting between CAR⁺/mPSCs and CAR⁺/mPSCs^{Oct-4}^{_}^{hi} clones (FIG. 6(A)). Table 4 showed the analysis of the population for G₁-, S-, and G₂/M-phases of cell cycle of CAR⁺/mPSCs and CAR⁺/mPSCs^{Oct-4}^{_}^{hi} C1, E9, and C7 clones. Both the S- and G₂/M-phase population of CAR⁺/mPSCs^{Oct-4}^{_}^{hi} clones were strongly increased.

TABLE 4
Analysis of G₁-, S-, and G₂/M-phases of cell cycle in
CAR⁺/mPSCs^{Oct-4}^{_{—}}^{hi} C1, E9, and C7 clones

| | | Cell cycle | | |
|---|---|---|---|---|
| | | G₁ | S | G₂/M |
| CAR⁺/mPSCs | | 88.0 ± 1.4 | 2.9 ± 0.5 | 9.2 ± 1.9 |
| CAR⁺/mPSCs^{Oct-4}^{_{—}}^{hi} | C1 | 67.2 ± 0.9 | 14.5 ± 1.2 | 16.6 ± 1.1 |
| clones** | E9 | 68.9 ± 1.0 | 14.6 ± 1.6 | 15.5 ± 0.6 |
| | C7 | 67.6 ± 1.3 | 14.3 ± 1.4 | 14.2 ± 1.9 |

Data are presented as mean ± SD.
**P < 0.01 compared with CAR+/mPSCs

Additionally, the C1, E9, and C7 clones could propagate for more than 50 passages, with a doubling time of 23±1 h (FIG. 6(B)). In table 5, it showed doubling time.

TABLE 5
Doubling time

| | | | Doubling Time (h) |
|---|---|---|---|
| CAR⁺/mPSCs | | | 63.5 ± 9.0 |
| | CAR⁺/mPSCs^{Oct-4}^{_{—}}^{hi} | C1 | 23.2 ± 1.6 |
| | clones | E9 | 23.1 ± 0.5 |
| | | C7 | 24.3 ± 0.7 |

Data are presented as mean ± SD

While telomerase activity was detected in the 12^{th}, 20^{th} and 50^{th} passages of the C1, E9, and C7 clones, it was not detected in CAR^{|}/mPSCs (FIG. 6(C)). These results demonstrated that Oct-4 hyperexpression in CAR⁺/mPSCs was sufficient to produce immortal effects, such as G₁ cell cycle progression, proliferation potential, and telomerase activity.

CAR⁺/mPSCs^{Oct-4}^{_}^{hi} Exhibited Tumorigenic Potential

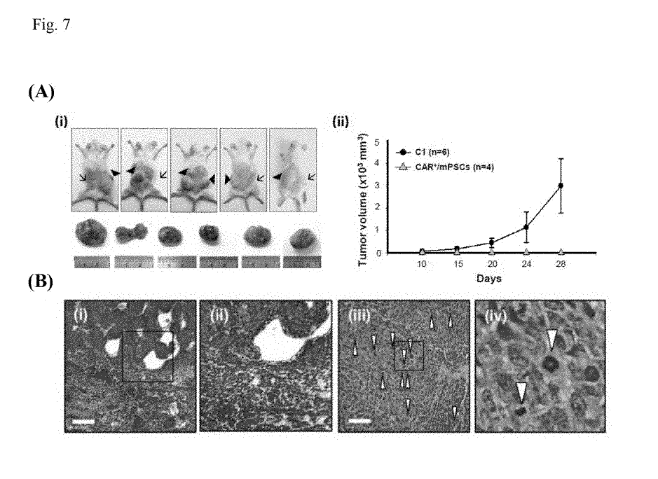

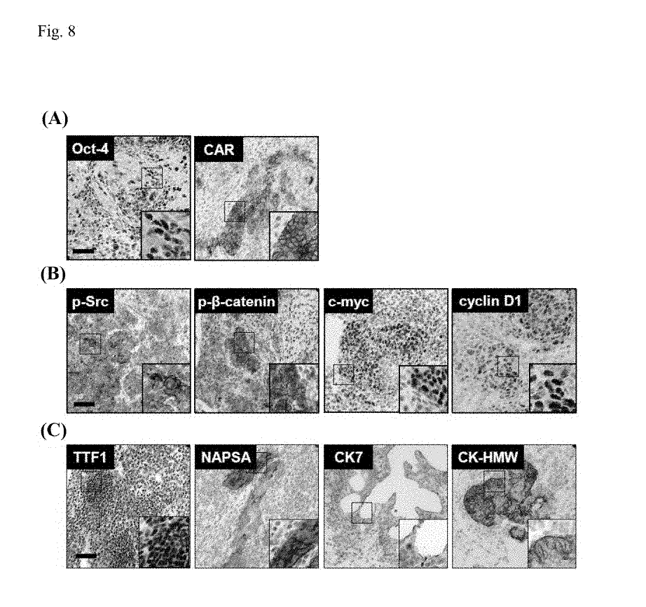

In order to evaluate the pluripotent potential of CAR^{|}/mPSCs^{Oct-4 hi} clones, teratoma formation assays were performed with the C1, E9, and C7 clones. 1×10⁶ cells of C1, E9, or C7 clones were subcutaneously implanted in SCID mice. After 20 to 24 d, teratomas of approximately 1 cm had developed (FIG. 7(A)). FIG. 7, B showed representative images of histopathological analysis of the C1 clone. Hematoxylin and eosin (H&E) staining showed that ectodermal, mesodermal, and endodermal lineage differentiation were absent in the tumors. Moreover, the tumors exhibited typical malignant phenotypic characteristics, such as a high cellular density; small, round immature cell proliferation; pleomorphic cells with a high nuclear/cytoplasmic ratio; and a high mitotic ratio (FIG. 7(B)). Using immunohistochemical staining, Oct-4 and CAR were detected in tumors (FIG. 8(A)). The active form of some oncogenes, including phospho-Src, phospho-β-catenin, c-myc, and cyclin D1 were also detected in the tumors (FIG. 8(B)). Lung adenocarcinoma diagnostic markers, such as thyroid transcription factor-1 (TTF1), Napsin A (NAPSA), cytokeratin 7 (CK7), and cytokeratin heavy molecular weight (CK-HMW) were also detected in the tumors (FIG. 8(C)). These data implied that CAR⁺/mPSCs acquired tumorigenic capacity through Oct-4 hyperexpression.

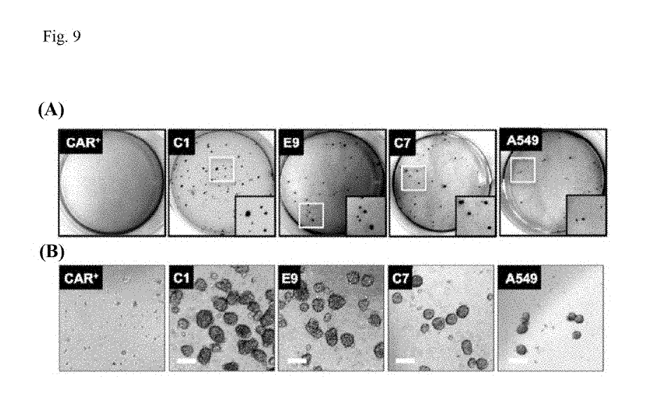

The tumorigenic potential of CAR⁺/mPSCs^{Oct-4}^{_}^{hi} clones was then quantified. Anchorage-independent growth was evaluated using a soft agar colony formation assay. After 2 weeks, the C1, E9, and C7 clones had formed more significant soft agar colonies number compared to the human lung adenocarcinoma cell line A549. However, no such colonies were observed for CAR⁺/mPSCs (FIG. 9(A) and table 6). To evaluate secondary sphere formation efficiency, C1, E9, and C7 clones were cultured under non-adhesion conditions. The secondary sphere forming efficiency of the CAR⁺/mPSCs^{Oct-4}^{_}^{hi} clones was significantly higher than that of A549 cells, and sphere formation was also absent in CAR^{|}/mPSCs (FIG. 9(B) and table 6). In table 6, it showed a quantification of colonies and spheres.

TABLE 6
Quantification of colonies and spheres

| | | Colonies number | Spheres number |
|---|---|---|---|
| CAR⁺/mPSCs | | 0 | 0 |
| CAR⁺/mPSCs^{Oct-4}^{_{—}}^{hi} | C1 | 256 ± 37 | 317 ± 83 |
| clones | E9 | 194 ± 22 | 170 ± 55 |
| | C7 | 192 ± 24 | 215 ± 73 |

| | | |
|---|---|---|
| A549 | 19 ± 4 | 96 ± 9 |

Data are presented as the mean ± SD

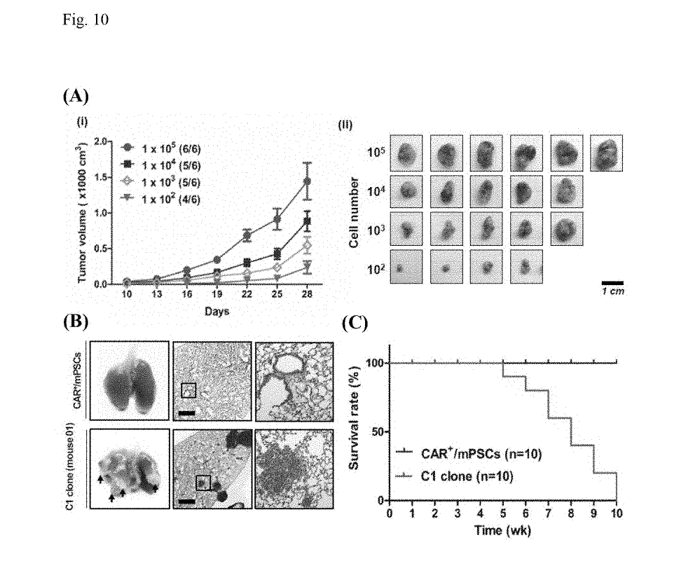

In the assays, the C1 clone exhibited the most pronounced tumorigenic behaviors, including the highest proliferation rate, highest efficiency in anchorage-independent colony formation, and secondary sphere generation; therefore, the C1 clone was selected for subsequent in vivo tumorigenic experiments. To determine the tumorigenicity of the C1 clone, a limiting dilution transplantation experiment was performed. Tumor formation potencies were 6/6, 5/6, and 5/6 in 10⁵, 10⁴, and 10³ cell concentrations of C1 clone injections, respectively. In addition, a low cell concentration of C1 clone (10²) was sufficient for tumor formation (4/6) at an average of 28 d after injection (FIG. 10(A)-i). Tumor size and morphology are shown in FIG. 10(A)-ii. In contrast, no tumor formation was observed in transplants using CAR^{|}/mPSCs (10⁶ cells) despite 56 d incubation (data not shown). In order to examine metastatic potential, 3×10⁵ cells of the C1 clone were transplanted through the tail vein of mice and allowed to develop for 35 d. All mice injected with the C1 clone developed tumor nodules in the lung tissue (FIG. 10(B)). H&E staining of lung tissue revealed extensive hemorrhage and nodule formation in the C1 clone transplants, while no abnormal lesions were detected after CAR⁺/mPSCs transplantation. Kaplan-Meier survival analysis was performed to determine the survival rate following C1 clone or CAR⁺/mPSCs transplantation. The mean survival of mice injected with the C1 clone was significantly lower than that of mice transplanted with CAR⁺/mPSCs (FIG. 10(C)). These results implied that CAR⁺/mPSCs undergo malignant transformation following Oct-4 hyperexpression, as evidenced by the in vitro and in vivo tumorigenic potential and tumor initiating capacity of the CAR⁺/mPSCs^{Oct-4}^{_}^{hi} clones.

CAR⁺/mPSCs^{Oct-4}^{_}^{hi} Exhibited Lung Cancer CICs Traits

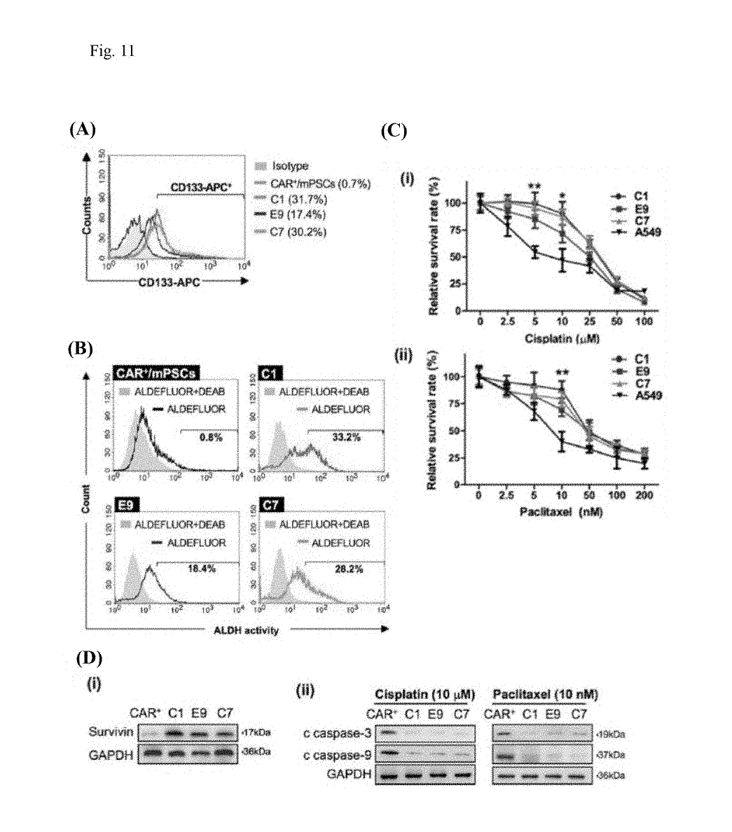

Different biomarkers for lung CICs have been proposed, including CD133 expression, ALDH activity, and chemoresistance. These biomarkers were utilized to further investigate CICs characteristics in CAR⁺/mPSCs^{Oct-4}^{_}^{hi} clones. Flow cytometry analysis revealed that about 17.4-31.7% of cells were CD133⁺ among the C1, E9, and C7 clones, whereas CD133⁺ cells were nearly undetectable in CAR⁺/mPSCs (FIG. 11(A)). ALDH activity was detected in 18.4-33.2% of the C1, E9, and C7 clones, whereas only 0.8% of CAR⁺/mPSCs exhibited ALDH activity (FIG. 11(B)). As chemoresistance is also a critical biological feature of CICs, the C1, E9, and C7 clones, chemoresistance to cisplatin or paclitaxel treatment was evaluated. The cell viability was shown in FIG. 11, C. In table 7, it showed IC₅₀ of cisplatin and paclitaxel for CAR⁺/mPSCs^{Oct-4}^{_}^{hi} C1, E9, and C7 clones and A549 cells. The IC₅₀ values of cisplatin were 26.4-34.7 μM for C1, E9. and C7 clones, indicating that CAR⁺/mPSCs^{Oct-4}^{_}^{hi} clones were about 2-3 fold more resistant to cisplatin compared to the A549 cells. For paclitaxel, the IC₅₀ were 43.2-47.2 nM in the C1, E9, and C7 clones, which were approximately 6 fold higher than that of the A549 cells (FIG. 11(C)).

TABLE 7
IC₅₀ of cisplatin and paclitaxel for CAR⁺/
mPSCs^{Oct-4}^{_{—}}^{hi} C1, E9, and C7 clones and A549 cells

| | IC₅₀ value measurement | | | |
|---|---|---|---|---|
| | CAR⁺/mPSCs^{Oct-4}^{_{—}}^{hi} clones | | | |
| | C1 | E9 | C7 | A549 |
| Cisplatin (μM) | 34.7 ± 1.4 | 26.4 ± 2.3 | 32.7 ± 1.6 | 13.5 ± 2.2 |
| Paclitaxel (nM) | 47.2 ± 2.0 | 43.2 ± 3.6 | 45.0 ± 2.5 | 8.2 ± 3.5 |

Data are shown as the mean ± SD

It has been well documented that the protein survivin inhibits apoptosis and plays an important role in conferring chemoresistance to CICs. The present invention found that survivin expression was significantly higher in the C1, E9, and C7 clones compared to CAR⁺/mPSCs (FIG. 11(D)). The C1, E9, and C7 clones also exhibited lower levels of cleaved caspase-3 and cleaved caspase-9 under cisplatin or paclitaxel treatment compared with CAR⁺/mPSCs (FIG. 11(D)-ii). Taken together, the results indicated that Oct-4 hyperexpression could drive the transformation of CAR⁺/mPSCs, conferring them with CICs-like properties.

Thus, the present invention further compared the differences of the CAR⁺/mPSCs, the CAR⁺/mPSCs^{Oct-4}^{_}^{hi} and the A549 cells (human lung adenocarcinoma cell line) (Table 3). Compared to the CAR^{|}/mPSCs. CAR⁺/mPSCs^{Oct-4}^{_}^{hi} had many traits of a cancer initiating cell. The A549 cells are cancer cells but not cancer initiating cells. Compared to the A549 cells, the CAR⁺/mPSCs^{Oct-4}^{_}^{hi} had higher expression levels of soft agar colony, sphere formation, CD133 and aldehyde dehydrogenase (ALDH) activity. Besides, a small number (10³ cells) of the CAR⁺/mPSCs^{Oct-4}^{_}^{hi} could form a tumor and had a tumor regeneration capacity. These data indicated the CAR⁺/mPSCs^{Oct-4}^{_}^{hi} was the cancer initiating cell.

TABLE 8
Comparison of CAR⁺/mPSCs, CAR⁺/
mPSCs^{Oct-4}^{_{—}}^{hi} and A549 cells

| | | | A549 |
|---|---|---|---|
| | | | (human lung |
| | CAR⁺/ | CAR⁺/ | adenocarcinoma |
| | mPSCs | mPSCs^{Oct-4}^{_{—}}^{hi} | cell line) |
| Immortalization | No | Yes | Yest |
| property | | | |
| Soft agar colony | No | 256-192 | 15-23 |
| formation | | | |
| (per 1000 cells) | | | |
| Sphere formation | No | 317-170 | 85-105 |
| CD133 | <1% | 17.4-30.2% | 0.3-1% |
| ALDH activity | <1% | 18.4-33.2% | 2-8% |
| Tumor formation | No | 5/6 | 0/3 |
| (incidence of 1000 | | | |
| cells) | | | |
| Metastasis capacity | No | 5/5 | 1/3 |
| Mortality rate in 10 | 0% | 100% | 40% |
| wk | | | |
| Tumor | — | 4/4 | — |
| regeneration | | | |
| efficiency | | | |
| Chemo-drug | — | IC50 of cisplatin: | IC50 of cisplatin: |
| resistance | | 26.4-34.7 μM | 13.5 μM |
| | | IC50 of paclitaxel: | IC50 of paclitaxel: |
| | | 43.2-47.2 nM | 8.2 nM |

CAR⁺/mPSCs^{Oct-4}^{_{—}}^{hi} participated in tumor angiogenesis

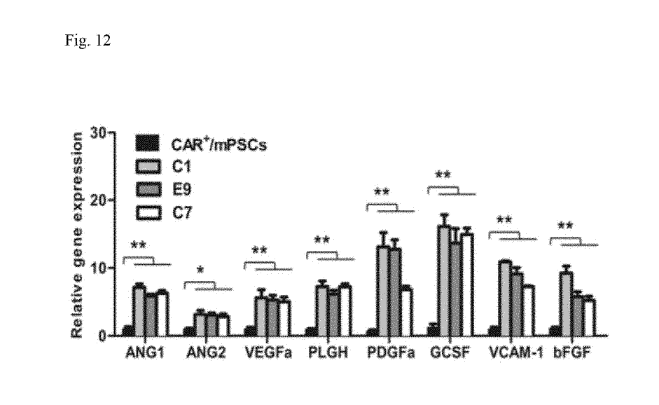

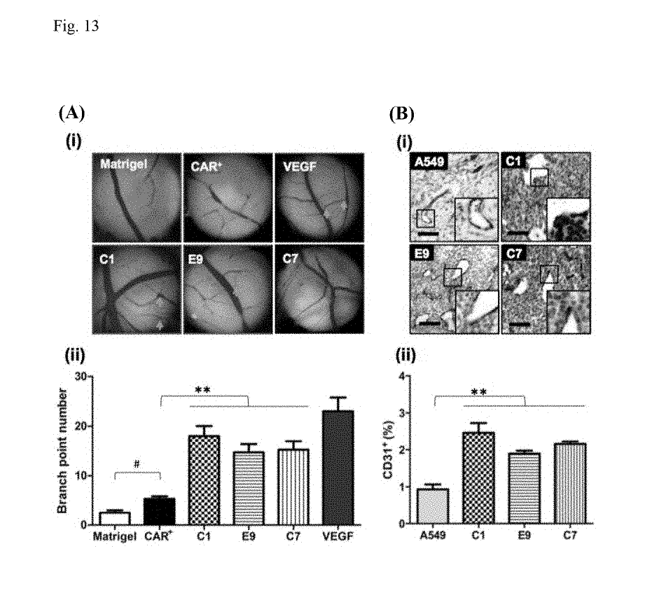

In the previous study, CAR⁺/mPSCs were shown to express the proangiogenic factors, including vascular endothelial growth factor A (VEGFa), granulocyte colony stimulating factor (GCSF), vascular cell adhesion molecule 1 (VCAM-1), and basic fibroblast growth factor (bFGF), which initiated endothelial cell tube formation. Therefore, the present invention wanted to evaluate the potential for angiogenesis in the CAR⁺/mPSCs^{Oct-4}^{_}^{hi} clones. Real-time PCR analysis was performed and the present invention found that proangiogenic factors, including angiopoietin 1 (ANG1), angiopoietin 2 (ANG2), VEGFa, placental growth factor (PLGF), platelet-derived growth factor A (PDGFa). GCSF, VCAM-1, and bFGF, were expressed at significantly higher levels in the C1, E9, and C7 clones compared with CAR⁺/mPSCs (FIG. 12). To further confirm angiogenic potential, we used the C1, E9, and C7 clones and CAR⁺/mPSCs in a CAM assay. When implanted on CAM, the C1, E9, and C7 clones induced extensive blood vessel formation compared with CAR⁺/mPSCs implants (FIG. 13(A)-i). Branch point quantification revealed that implanting the C1, E9, and C7 clones significantly increased blood vessel branching compared with that of CAR⁺/mPSCs (FIG. 13(A)-ii). Immunohistochemical analysis and quantification of the C1, E9, and C7 clone-derived tumors revealed a significantly higher CD31-positive population than that found in A549 derived tumors (FIG. 13(B)-i and -ii). These data implied that Oct-4 hyperexpression enhanced the angiogenic potential in CAR⁺/mPSCs^{Oct-4}^{_}^{hi} clones.

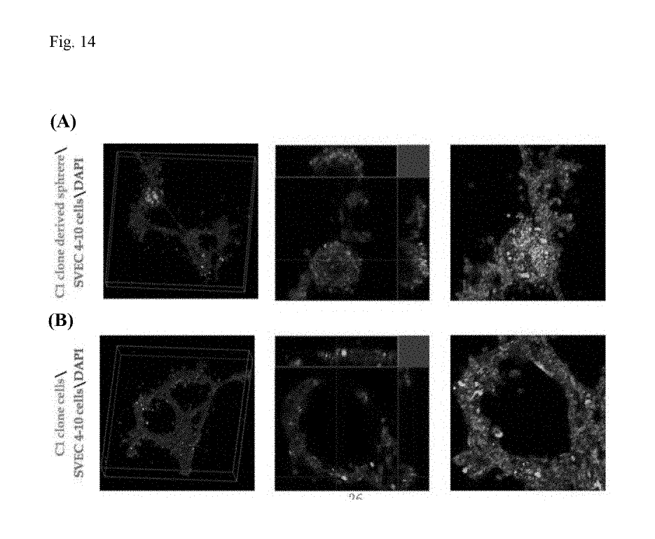

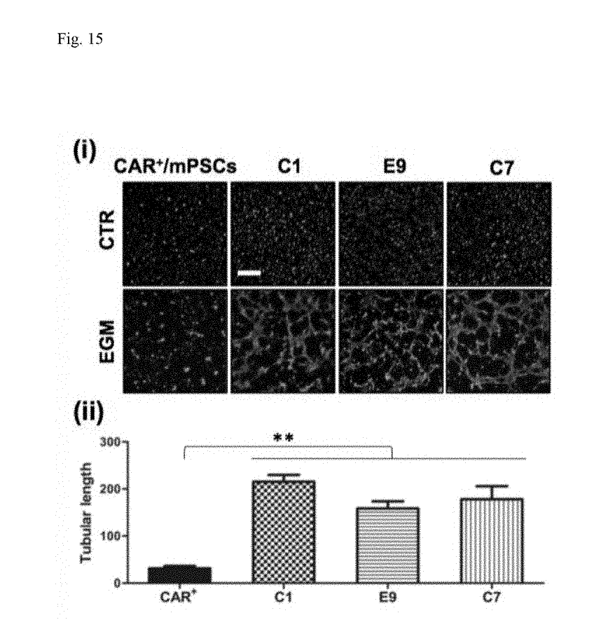

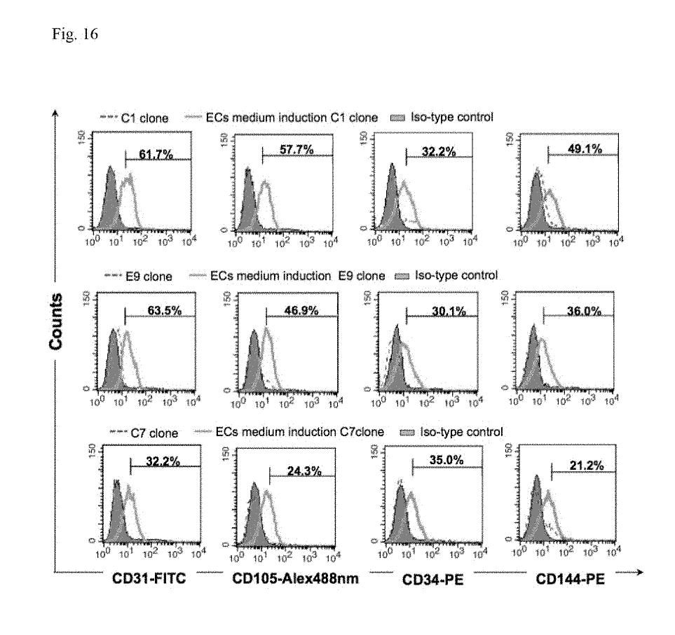

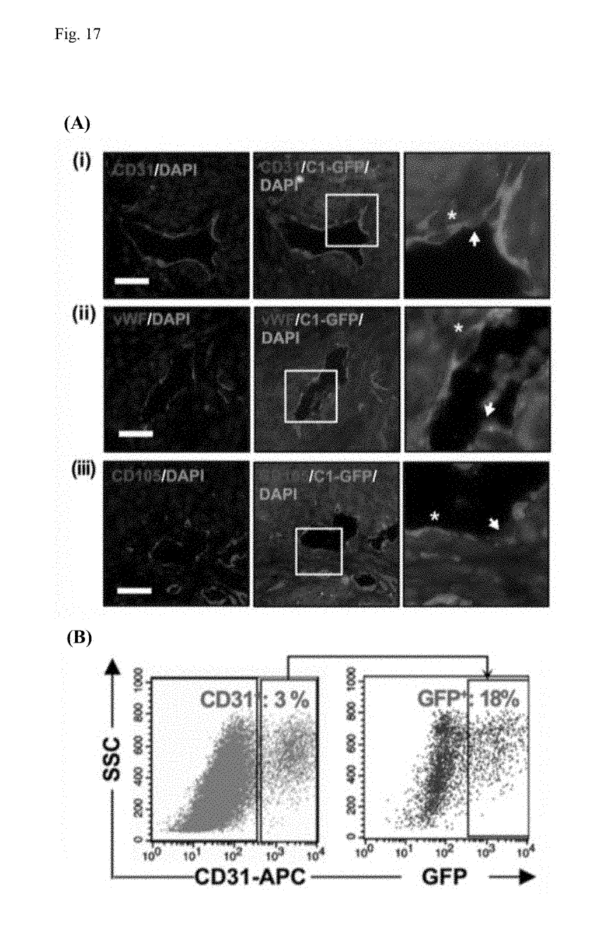

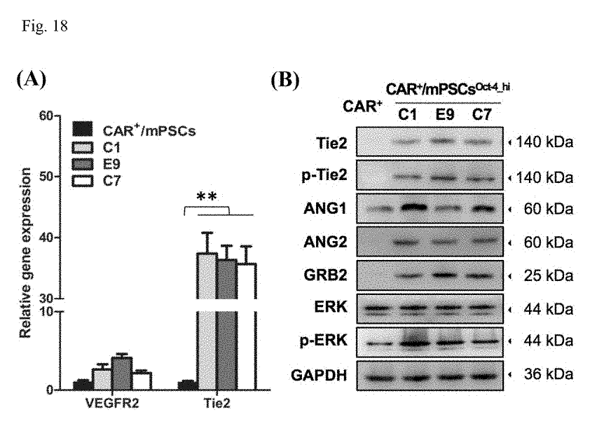

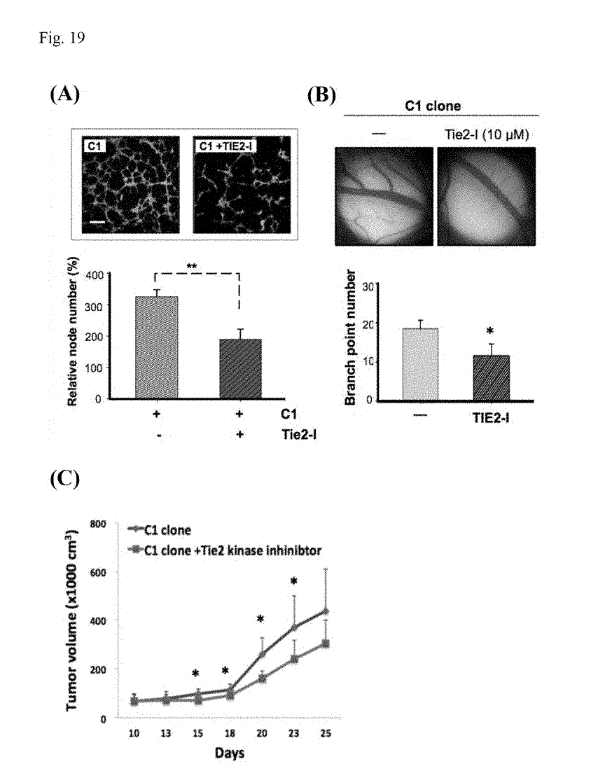

To further elucidate the functional contribution of CAR⁺/mPSCs^{Oct-4}^{_}^{hi} in angiogenesis, tube formation of endothelial cells after incubation with the C1 clone was monitored. C1 clone-derived spheres labeled with the green fluorescent tracer, calcein-AM, were mixed with SVEC4-10 cells that had been labeled with the red fluorescent tracer, PKH26, and then were co-cultured for tube formation. C1 clone-derived spheres recruited SVEC4-10 cells and established tube network (FIG. 14(A)). Some C1 clone cells were observed to integrate into the SVEC4-10 cells tube network (FIG. 14(B)). The C1, E9, and C7 clones in an endothelial cell growth medium (EGM; Lonza) were then cultured for 7 d to examine tube formation ability; CAR⁺/mPSCs were also cultured as a control. EGM cultured C1, E9, and C7 clones exhibited tube formation ability, whereas no such capability was observed with CAR⁺/mPSCs cultured in EGM (FIG. 15(i) and (ii)). In EGM culture, C1, E9, and C7 clones exhibited the endothelial cells markers expression, including CD31, CD105, CD34, and CD144 (FIG. 16) These results indicated that CAR⁺/mPSCs^{Oct-4}^{_}^{hi} clones not only possessed angiogenic potential, but were also involved in tube formation in vitro. To evaluate tumor blood vessel formation potential in vivo, the C1 clone was transfected with GFP expression (C1-GFP clone) and then used to form tumors via subcutaneous implantation in SCID mice. Using immunofluorescence staining, the endothelial antigens, including CD31, vWF, and CD105, were detected in C1-GFP clone-derived tumors. Some blood vessels incompletely expressed the endothelial antigens, and GFP⁺ cells were directly integrated into blood vessels, exhibiting a mosaic-like pattern. Moreover, some endothelial cells simultaneously expressed endothelial antigens and were GFP-positive (FIG. 17(A)-i, ii and iii). To confirm the presence of GFP⁺-endothelial cells, dissociated tumors were examined using flow cytometry. Similar to the results of immunofluorescence staining, 12-18% of the CD31⁺ population was also positive for GFP (FIG. 17(B)). In order to examine the correlation between CAR⁺/mPSCs^{Oct-4}^{_}^{hi} clones and endothelial cells, real-time PCR was used to analyze gene expression of the angiogenesis associated receptors, VEGF receptor 2 (VEGFR2) and Tie2. The receptor, Tie2, which was specifically expressed in endothelial cells, was significantly elevated in EGM cultured C1, E9, and C7 clones compared with CAR⁺/mPSCs, while gene expression of VEGFR2 showed no significant difference (FIG. 18(A)). Western blot analysis confirmed the involvement of the ANGs/Tie2/GRB2/ERK signaling pathway, showing that ANG1, ANG2, phospho-Tie2, GRB2, and phospho-ERK expression were significantly increased in EGM cultured C1, E9, and C7 clones relative to CAR⁺/mPSCs (FIG. 18(B)). These data implied that CAR⁺/mPSCs^{Oct-4}^{_}^{hi} actively participated in tumor angiogenesis, rather than playing a passive role as CAR⁺/mPSCs do. Further, Tie2 kinase inhibitor reduced the tube formation potential of EGM cultured C1 clone and inhibited the blood vessel formation of C1 clone promotion in CAM assay (FIG. 19(A)). In addition, there was no significant difference in tube formation of EGM cultured C1 clone between antibody neutralized VEGFA group and IgG control group (FIG. 19(B)). In xenograft tumor assay, 50 mg/kg BW of Tie2 inhibitor were administrated through intraperitoneal injection once every two days from 7 days after C1 clone transplantation. Tie2 inhibitor significantly reduced the tumor volume of C1 at early phase of tumor development (FIG. 19(C)). Thus, the present invention proposed that the mechanism of tumor blood vessel formation might be different from that of regular tumor vascular formation. Tie2/ANGs signaling played important role in the CAR⁺/mPSCs^{Oct-4}^{_}^{hi} clone actively participated in tumor angiogenesis.

One skilled in the art readily appreciates that the present invention is well adapted to carry out the objects and obtain the ends and advantages mentioned, as well as those inherent therein. The cells, method of creating the same, and uses thereof are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A cancer initiating cell comprises an isolated coxsackievirus and adenovirus receptor positive mouse pulmonary stem/progenitor cell (CAR^{|}/mPSC) that overexpresses Oct-4,

2. The cancer initiating cell of claim 1, wherein the CAR⁺/mPSC comprises a vector for encoding an Oct-4 gene.

3. The cancer initiating cell of claim 2, wherein the sequence of the Oct-4 gene is SEQ ID NO: 1.

4. The cancer initiating cell of claim 1, wherein the expression level of the Oct-4 in the CAR⁺/mPSC is 16 times higher than that of an normal CAR⁺/mPSC.

5. The cancer initiating cell of claim 1, which is a lung cancer initiating cell.

6. The cancer initiating cell of claim 1, which has tumorigenic capacity, wherein the tumorigenic capacity comprises tumor formation, tumor regeneration, metastatic capacity or combination thereof.

7. The cancer initiating cell of claim 1, which exhibits a CD 133 expression, an aldehyde dehydrogenase (ALDH) activity, a chemoresistance or combination thereof.

8. The cancer initiating cell of claim 1, which has a function for angiogenesis.

9. The cancer initiating cell of claim 1, which has a function for participating in tumor blood vessel formation

10. The cancer initiating cell of claim 1, which expresses a surface marker of an endothelial cell, wherein the surface marker of the endothelial cell comprises CD31, CD105, CD34. CD144 or combination thereof.

11. The cancer initiating cell of claim 8, which has a function for activating ANG/Tie2 signal pathway to enhance the angiogenesis.
